(19) United States
(12) Patent Application Publication
(10) Pub. No.: US 2006/0128684 A1
(43) Pub. Date: Jun. 15, 2006

(54) Anthranilic acid amide derivatives and their pharmaceutical use

(76) Inventors: Guido Bold, GIPF-OBERFRICK (CH); Pascal Furet, Thann (FR); Paul William Manley, Arlesheim (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/538,199

(22) PCT Filed: Dec. 11, 2003
(86) PCT No.: PCT/EP03/14086

§ 371 Date: Jun. 9, 2005

(30) Foreign Application Priority Data
Dec. 12, 2002 (GB) 0229022.9

(51) Int. Cl. A61K 31/5377; A61K 31/496; A61K 31/444; A61K 31/4415; C07D 417/02; C07D 409/02; C07D 403/02

(52) U.S. Cl. 514/210.2; 544/360; 546/268.4; 546/297; 544/124; 514/235.2; 514/332; 514/349; 514/253.11

(57) ABSTRACT

The present invention relates to new anthranilic acid amide derivatives, processes for the preparation thereof, the application thereof in a process for the treatment of the human or animal body, the use thereof—alone or in combination with one or more other pharmaceutically active compounds.

The invention relates to new anthranilic acid amide derivatives, processes for the preparation thereof, the application thereof in a process for the treatment of the human or animal body, the use thereof—alone or in combination with one or more other pharmaceutically active compounds—for the treatment especially of a neoplastic disease, such as a tumor disease, of retinopathy and age-related macular degeneration; a method for the treatment of such a disease in animals, especially In humans, and the use of such a compound—alone or in combination with one or more other pharmaceutically active compounds—for the manufacture of a pharmaceutical preparation for the treatment of a neoplastic disease, of retinopathy or age-related macular degeneration.

Certain diseases are known to be associated with deregulated angiogenesis, for example diseases caused by ocular neovascularisation, such as retinopathies (including diabetic retinopathy), age-related macular degeneration, psoriasis, haemangiob)astoma, haeman-gioma, arteriosclerosis, inflammatory diseases, such as rheumatoid or rheumatic inflammatory diseases, especially arthritis, such as rheumatoid arthritis, or other chronic inflammatory disorders, such as chronic asthma, arterial or post-transplantational atherosclerosis, endometriosis, and especially neoplastic diseases, for example so-called solid tumours and liquid tumouTs (such as leucemias).

At the centre of the network regulating the growth and differentiation of the vascular system and its components during embryonic development, normal growth and in a wide number of pathological anomalies and diseases, lies the angiogenic factor known as “Vascular Endothelial Growth Factor” (VGEF), a dimeric, disulfide-linked 46-kDa glycoprotein, along with its cellular receptors (see Breier, G., et al., Trends In Cell Biology 6, 454-6[1996)). VEGF receptors are transmembranous receptor tyrosine kinases. Various types of VEGF receptor are known, e.g. VEGFR-1, VEGFR-2, and VEGFR-3.

A large number of human tumors, especially gliomas and carcinomas, express high levels of VEGF and its receptors. This has led to the hypothesis that the VEGF released by tumor cells could stimulate the growth of blood capillaries and the proliferation of tumor endothelium in a paracrine manner and thus, through the improved blood supply, accelerate tumor growth. Direct evidence of the role of VEGF as a tumor angiogenesis factor in vivo has been obtained from studies in which VEGF activity was inhibited by antibodies.

Angiogenesis is regarded as an absolute prerequisite for those tumors which grow beyond a maximum diameter of about 1-2 mm; up to this limit, oxygen and nutrients may be supplied to the tumor cells by diffusion.

Three principal mechanisms play an important part in the activity of angiogenesis inhibitors against tumors: 1) inhibition of the growth of vessels, especially capillaries, into a vascular resting tumors, with the result that there is no net tumor growth; 2) prevention of the migration of tumor cells owing to the absence of blood flow to and from tumors; and 3) inhibition of endothelial cell proliferation, thus avoiding the paracrine growth-stimulating effect exerted on the surrounding tissue by the endothelial cells which normally line the vessels.

In WO00/27820 and WO 01/55114 compounds are described belonging to the class of anthranilic acid amides which are reported to Inhibit the activity of the VEGF receptor tyrosine kinase, the growth of tumors and VEGF-dependent cell proliferation.

Surprisingly, it has now been found that the anthranilic acid amide derivatives of formula I, described below, have advantageous pharmacological properties and inhibit, for example, the activity of the VEGF receptor tyrosine kinase, the growth of tumors and VEGF-dependent cell proliferation.

The anthranilic acid amide derivatives of formula I are suitable, for example, to be used in the treatment of diseases, especially for diseases In the treatment and also for the prevention of which an inhibition of anglogenesis and/or of the VEGF receptor tyrosine kinase shows beneficial effects.

The invention pertains to anthranilic acid amides of formula I,

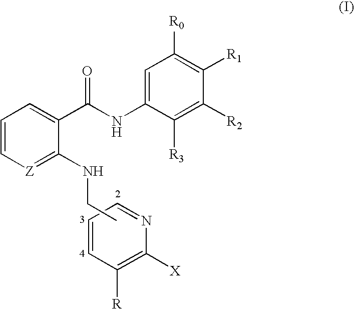

wherein
R and R₀ represent H, halogen,
   alkynyl, alkenyl, alkyl, which in each case is unsubstituted or substituted by halogen;
   unsubstituted or substituted mono- or bicyclic aryl;
   unsubstituted or substituted mono- or bicyclic heteroaryl having 1 to 3 heteroatoms selected from O, N or S;
   unsubstituted or substituted heterocyclyl having at least one N atom; mono- or dialkyl amino, wherein the alkyl radical is unsubstituted or substituted by unsubstituted or substituted aryl, unsubstituted or substituted mono- or bicyclic heteroaryl having 1 to 3 heteroatoms selected from O, N or S or substituted by unsubstituted or substituted heterocyclyl having at least one N atom;
   unsubstituted or substituted heterocyclyl carbonyl alkyl amino, wherein the heterocyclyl radical comprises at least one N atom;

R₁ represents H, halogen, unsubstituted or substituted C₁₋₇alkyl, C₂₋₇alkenyl, C₂₋₇alkynyl, alkoxy or a radical —O—(CH₂)ₙ-CF₃, wherein n is 0, 1, 2 or 3,

R₂ is perfluoro alkyl,

R₃ represents H or halogen,

X represents hydroxy, alkoxy, alkyl thio, imino, alkyl imino, halogen, a radical of formula I′ 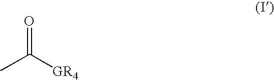
wherein G is CH₂ or NH and R₄ is hydrogen, alkyl or aryl, or a radical of formula I″ 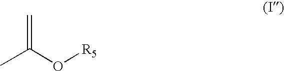
wherein R₅ is alkyl or aryl,

Z is N or CH, and
wherein the methylen group is attached to the pyridyl moiety at the carbon atom of the pyridyl moiety in 2-, 3-, 4- or 5-position,
under the proviso that R cannot represent H, if Z is nitrogen, X is hydroxy or methoxy and wherein the methylen group is attached to the pyridyl moiety at the carbon atom of the pyridyl moiety in 3-position, and R₁ and R₃ cannot both represent H if Z is CH, R represents H, X is hydroxy, alkoxy or alkyl thio and wherein the methylen group is attached to the pyridyl moiety at the carbon atom of the pyridyl moiety in 3-position,
   to the N-oxides and tautomers thereof,
   and to the salts of such anthranilic acid amides, their N-oxides and tautomers.

The general terms used hereinbefore and hereinafter preferably have within the context of this disclosure the following meanings, unless otherwise indicated:

The prefix “lower” denotes a radical having up to and including a maximum of 7, especially up to and including a maximum of 4 carbon atoms, the radicals in question being either linear or branched with single or multiple branching,

Where the plural form is used for compounds, salts, and the like, this is taken to mean also a single compound, salt, or the like.

The invention relates also to possible tautomers of the compounds of formula 1. The term “tautomers” as used herein relates in particular to compounds of formula I wherein X is hydroxy, which compounds does also exist to some extend, if not totally, in the tautomeric form shown below (I—Tautomer), wherein the further radicals and symbols have the meaning as defined herein.

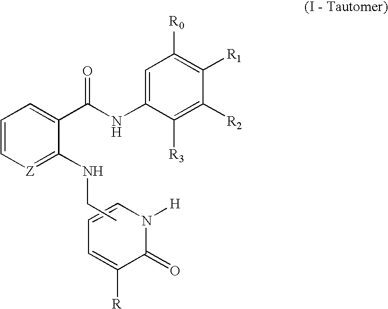

In a preferred embodiment, alkyl, alkenyl and alkanoyl have up to a maximum of 12 carbon atoms and are especially lower alkyl, lower alkenyl and lower alkanoyl.

Lower alkyl is preferably alkyl with from and including 1 up to and including 7, preferably from and including 1 to and including 4, and is linear or branched; preferably, lower alkyl is butyl, such as n-butyl, sec-butyl, isobutyl, tert-butyl, propyl, such as n-propyl or isopropyl, ethyl or preferably methyl.

Alkenyl is preferably lower alkenyl. Lower alkenyl is preferably alkenyl with from and including 2 up to and including 7, preferably from and including 2 to and including 5, and is linear or branched; preferably, lower alkenyl is allyl, butenyl, e.g. 2-butenyl, or methyl-butenyl, e.g. 3-methyl-2-butenyl.

Lower alkanoyl is preferably alkanoyl with from and including 1 up to and including 7, preferably from and including 1 to and including 4, and Is linear or branched; preferably, lower alkanoyl is formyl or acetyl.

Alkynyl Is preferably C₂-₇alkynyl, in particular ethynyl, propynyl or 2-butynyl, especially propynyl.

Alkyl which is substituted by halogen is preferably perfluoro alkyl.

The term “perfluoro alkyl” as used herein means an alkyl radical wherein all hydrogen atoms are replaced by fluoro atoms, and is preferably perfluoro lower alkyl, in particular trifluoro-methyl.

Preferably, the methylen group is attached to the pyridyl moiety at the carbon atom of the pyridyl moiety in 3-position.

Halogen is especially fluorine, chlorine, bromine, or iodine, especially fluorine, chlorine, or bromine. “Aryl” is an aromatic radical which is bound to the molecule via a bond located at an aromatic ring carbon atom of the radical. In a preferred embodiment, aryl is an aromatic radical having 6 to 14 carbon atoms, and denotes especially monocyclic aryl, in particular phenyl, or bicyclic aryl, in particular naphthyl, tetrahydronaphthyl, fluorenyl or phenanthrenyl, and is unsubstituted or substituted by one or more, preferably up to three, especially one or two substituents, especially selected from amino, mono- or disubstituted amino, halogen, lower alkyl, substituted alkyl, lower alkenyl, lower alkynyl, hydroxy, etherified or esterified hydroxy, nitro, cyano, carboxy, esterdfied carboxy, alkanoyl, benzoyl, carbamoyl, N-mono- or N,N-dialkyl carbamoyl, amidino, guanidino, mercapto, lower alkylthio, phenyl, phenoxy, phenylthio, lower alkanesulfonyl and phenylsulfonyl. Aryl is more preferably phenyl or naphthyl, which in each case is either unsubstituted or independently substituted by one or two substituents selected from the group comprising halogen, especially fluorine, chlorine, or bromine; hydroxy; hydroxy, etherified by lower alkyl, e.g. methyl or by halogen-lower alkyl, e.g. trifluoromethyl; lower alkyl, e.g. methyl or propyl; lower alkynyl, such as 1-propynyl; esterified carboxy, especially lower alkoxy carbonyl, e.g. methoxy carbonyl, n-propoxy carbonyl or iso-propoxy carbonyl; N-mono-substituted carbamoyl, in particular carbamoyl monosubstituted by lower alkyl, e.g. methyl, n-propyl or iso-propyl; substituted alkyl, especially lower alkyl, e.g. methyl or ethyl, substituted by lower alkoxy carbonyl, e.g. methoxy carbonyl or ethoxy carbonyl; lower alkanoyl amino, e.g. acetylamino; lower alkoxy phenyl; and halogen-lower alkyl, e.g. trifluoromethyl. “Heterocyclyl having at least one nitrogen atom” is especially a mono- or bicyclic group with one nitrogen atom and zero or one further heteroatom selected from the group consisting of nitrogen, oxygen, and sulfur, which may be wholly or partly saturated, and can be unsubstituted or substituted by the substituents listed for “aryl” above. Heterocyclyl is especially piperidinyl, e.g. 4-piperidinyl, morpholinyl, e.g. 4-morpholinyl, or piperazinyl, which is unsubstituted or substituted by lower alkyl, e.g. N-methyl-piperazinyl. “Unsubstituted or substituted mono- or bicyclic heteroaryl having 1 to 3 heteroatoms selected from O, N or S”, is in particular pyridyl, quinolyl, isoquinolyl, benzothienyl, benzofuranyl, benzopyranyl, benzothiopyranyl, furanyl, pyrrolyl, thiazolyl, oxazolyi, isoxazolyl, triazolyl, tetrazolyi, pyrrazolyl, imidazolyl or thienyl, or any said radical substituted by trifluoromethyl, phenyl, alkanoyl, alkanoyl amino, lower alkyl or halogen. “Etherified hydroxy” is especially lower alkoxy (preferred), such as methoxy, ethoxy, isopropyloxy, or n-pentyloxy, phenyl-lower alkoxy, such as benzyloxy, or also phenyloxy, or halogen-lower alkoxy, such as trifluoromethyloxy or 1,1,2,2-tetrafluoroethoxy. “Esterified hydroxy” is especially lower alkanoyloxy, benzoyloxy, lower alkoxycarbonyloxy, such as tert-butoxycarbonyloxy, or phenyl-lower alkoxycarbonyloxy, such as benzyloxycarbonyloxy.

“Esterified carboxy” is especially lower alkoxycarbonyl, such as tert-butoxycarbonyl, isopropoxycarbonyl, methoxycarbonyl or ethoxycarbonyl, phenyl-lower alkoxycarbonyl, or phenyloxycarbonyl.

In view of the close relationship between the novel compounds in free form and those in the form of their salts, including those salts that can be used as intermediates, for example in the purification or Identification of the novel compounds, any reference to the free compounds hereinbefore and hereinafter is to be understood as referring also to the corresponding salts, as appropriate and expedient.

Salts are formed, for example, as acid addition salts, preferably with organic or inorganic acids, from compounds of formula I with a basic nitrogen atom, especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, proplonic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred.

The compounds of formula I and N-oxides thereof have valuable pharmacological properties, as described hereinbefore and hereinafter.

The efficacy of the compounds of the invention as inhibitors of VEGF-receptor tyrosine kinase activity can be demonstrated as follows:

Test for activity against VEGF-receptor tyrosine kinase. The test is conducted using Flt-1 VEGF-receptor tyrosine kinase. The detailed procedure is as follows: 30 μl kinase solution (10 ng of the kinase domain of Flt-1, Shibuya et al., Oncogene 5, 519-24[1990]) in 20 mM Tris.HCI pH 7.5, 3 mM manganese dichloride (MnCl₂), 3 mM magnesium chloride (MgCl₂), 10 μM sodium vanadate, 0.25 mg/ml polyethylenglycol (PEG) 20000, 1 mM dithiothreitol and 3 μg/μl poly(Glu,Tyr) 4:1 (Sigma, Buchs, Switzerland), 8 μM [³³P]-ATP (0.2 μCi), 1% dimethyl sulfoxide, and 0 to 100 μM of the compound to be tested are incubated together for 10 minutes at room temperature. The reaction is then terminated by the addition of 10 μl 0.25 M ethylenediaminetetraacetate (EDTA) pH 7. Using a multichannel dispenser (LAB SYSTEMS, USA), an aliquot of 20 μl is applied to a PVDF (=polyvinyl difluoride) immobilon P membrane (Millipore, USA), through a Millipore microtiter filter manifold and connected to a vacuum. Following complete elimination of the liquid, the membrane is washed 4 times successively in a bath containing 0.5% phosphoric acid (H₃PO₄) and once with ethanol,

Incubated for 10 minutes each time while shaking, then mounted in a Hewlett Packard TopCount Manifold and the radioactivity measured after the addition of 10 μl Microscint®) (β-scintillation counter liquid). IC₅₀-values are determined by linear regression analysis of the percentages for the inhibition of each compound in three concentrations (as a rule 0.01, 0.1, and 1 μmol). The IC₅₀-values that can be found with compounds of formula I are in the range of 0.001 to 20 μM, preferably in the range from 0.001 to 0.1 μM.

The efficacy of the compounds of the invention as inhibitors of c-Abl protein-tyrosine kinase activity can be demonstrated as follows:

An in vitro enzyme assay is performed in 96-well plates as a filter binding assay as described by Geissler et al. in Cancer Res. 1992; 52:4492-4498, with the following modifications. The His-tagged kinase domain of c-Abl is cloned and expressed in the baculovirus/Sf9 system as described by Bhat et at in J.Biol.Chem. 1997; 272:16170-16175. A protein of 37 kD (c-Abl kinase) is purified by a two-step procedure over a Cobalt metal chelate column followed by an anion exchange column with a yield of 1-2 mg/L of Sf9 cells (Bhat et al., reference cited). The purity of the c-AbI kinase is >90% as judged by SDS-PAGE after Coomassie blue staining. The assay contains (total volume of 30 μL): c-Abl kinase (50 ng), 20 mM Tris.HCI, pH 7.5, 10 mM MgCl₂, 10 μM Na₃VO₄, 1 mM DTT and 0.06 μCi/assay [y³³P]-ATP (5 μM ATP) using 30 μg/mL poly-Ala,Glu,Lys,Tyr-6:2:5:1 (Poly-AEKY, Sigma P1152) in the presence of 1% DMSO. Reactions are terminated by adding 10 μL of 250 mM EDTA and 30 μL of the reaction mixture is transferred onto immobilon-PVDF membrane (Millipore, Bedford, Mass., USA) previously soaked for 5 min with methanol, rinsed with water, then soaked for 5 min with 0.5 % H₃PO₄ and mounted on vacuum manifold with disconnected vacuum source. After spotting all samples, vacuum is connected and each well rinsed with 200 μL 0.5% H₃PO₄. Membranes are removed and washed on a shaker with 0.5% H₃PO₄ (4 times) and once with ethanol. Membranes are counted after drying at ambient temperature, mounting In Packard TopCount 96-well frame, and addition of 10 μL/well of Microscint TM (Packard).

The antitumor efficacy of the compounds of the invention can be demonstrated in vivo as follows:

In vivo activity in the nude mouse xenotransplant model: female BALB/c nude mice (8-12 weeks old), Novartis Animal Farm, Sisseln, Switzerland) are kept under sterile conditions with water and feed ad libitum. Tumors are induced either by subcutaneous injecton of tumor cells into mice (for example, Du 145 prostate carcinoma cell line (ATCC No. HTB 81; see Cancer Research 37, 4049-58 (1978)) or by implanting tumor fragments (about 25 mg) subcutaneously into the left flank of mice using a 13-gauge trocar needle under Forene® anaesthesia (Abbott, Switzerland). Treatment with the test compound is started as soon as the tumor has reached a mean volume of 100 mm³. Tumor growth is measured two to three times a week and 24 hours after the last treatment by determining the length of two perpendicular axes. The tumor volumes are calculated In accordance with published methods (see Evans et al., Brit. J. Cancer 45, 466-8 [1982]). The antitumor efficacy is determined as the mean increase in tumor volume of the treated animals divided by the mean increase in tumor volume of the untreated animals (controls) and, after multiplication by 100, is expressed as T/C%. Tumor regression (given in %) is reported as the smallest mean tumor volume in relation to the mean tumor volume at the start of treatment. The test compound is administered daily by gavage.

As an alternative other cell lines may also be used in the same manner, for example:
the MCF-7 breast adenocarcinoma cell line (ATCC No. HTB 22; see also J. Natl. Cancer Inst. (Bethesda) 51, 1409-16 [1973]);
the MDA-MB 468 breast adenocarcinoma cell line (ATCC No. HTB 132; see also In Vitro 14, 911-15 [1978]);
the MDA-MB 231 breast adenocarcinoma cell line (ATCC No. HTB 26; see also J. NatI. Cancer Inst. (Bethesda) 53, 661-74 [1974]);
the Colo 205 colon carcinoma cell line (ATCC No. CCL 222; see also Cancer Res. 38, 1345-55 [1978]);
the HCT 116 colon carcinoma cell line (ATCC No. CCL 247; see also Cancer Res. 41, 1751-6 [1981]);
the DU145 prostate carcinoma cell line DU 145 (ATCC No. HTB 81; see also Cancer Res. 37, 4049-58 (1978]); and
the PC-3 prostate carcinoma cell line PC-3 (ATCC No. CRL 1435; see also Cancer Res. 40, 524-34 [1980]).

The inhibition of VEGF-induced KDR-receptor autophosphorylation can be confirmed with a further in vitro experiment in cells: transfected CHO cells, which permanently express human VEGF receptor (KDR), are seeded in complete culture medium (with 10% fetal calf serum=FCS) in 6-well cell-culture plates and incubated at 37° C. under 5% CO₂ until they show about 80% confluency. The compounds to be tested are then diluted in culture medium (without FCS, with 0.1% bovine serum albumin) and added to the cells. (Controls comprise medium without test compounds). After two hours' incubation at 37° C., recombinant VEGF is added; the final VEGF concentration is 20 ng/ml). After a further five minutes' incubation at 37° C., the cells are washed twice with ice-cold PBS (phosphate-buffered saline) and immediately lysed in 100 μl lysis buffer per well. The lysates are then centrifuged to remove the cell nuclei, and the protein concentrations of the supernatants are determined using a commercial protein assay (BIORAD). The lysates can then either be immediately used or, if necessary, stored at −20° C.

A sandwich ELISA is carried out to measure the KDR-receptor phosphorylation: a monoclonal antibody to KDR (for example Mab 1495.12.14; prepared by H. Towbin) is immobilized on black ELISA plates (OptiPlate™ HTRF-96 from Packard). The plates are then washed and the remaining free protein-binding sites are saturated with 1% BSA in PBS. The cell lysates (20 μg protein per well) are then incubated In these plates overnight at 4° C. together with an anti-phosphotyrosine antibody coupled with alkaline phosphatase (PY20:AP from Transduction Laboratories). The (plates are washed again and the) binding of the antiphosphotyrosine antibody to the captured phosphorylated receptor is then demonstrated using a luminescent AP substrate (CDP-Star, ready to use, with Emerald II; TROPIX). The luminescence is measured in a Packard Top Count Microplate Scintillation Counter (Top Count). The difference between the signal of the positive control (stimulated with VEGF) and that of the negative control (not stimulated with VEGF) corresponds to VEGF-induced KDR-receptor phosphorylation (=100%). The activity of the tested substances Is calculated as % inhibition of VEGF-induced KDR-receptor phosphorylation, wherein the concentration of substance that induces half the maximum inhibition is defined as the ED50 (effective dose for 50% inhibition).

A compound of formula I or a N-oxide thereof inhibits to varying degrees also other tyrosine kinases involved in signal transduction which are mediated by trophic factors, for example kinases from the Src family, especially c-Src kinase, Lck, and Fyn; also kinases of the EGF family, for example, c-erbB2 kinase (HER-2), c-erbB3 kinase, c-erbB4 kinase; insulin-like growth factor receptor kinase (IGF-1 kinase), especially members of the PDGF-receptor tyrosine kinase family, such as PDGF-receptor kinase, CSF-1-receptor kinase, Kit-receptor kinase and VEGF-receptor kinase; and also serine/threonine kinases, all of which play a role in growth regulation and transformation in mammalian cells, including human cells.

On the basis of these studies, a compound of formula I according to the invention shows therapeutic efficacy especially against disorders dependent on protein kinase, especially proliferative diseases.

The usefulness of a compound of the formula I in the treatment of arthritis as an example of an inflammatory rheumatic or rheumatoid disease can be demonstrated as follows:

The well-known rat adjuvant arthritis model (Pearson, Proc. Soc. Exp. Biol. 91, 95-101 (1956)) is used to test the anti-arthritic activity of compounds of the formula 1, or salts thereof. Adjuvant Arthritis can be treated using two different dosing schedules: either (i) starting time of immunisation with adjuvant (prophylactic dosing); or from day 15 when the arthritic response is already established (therapeutic dosing). Preferably a therapeutic dosing schedule is used. For comparison, a cyclooxygenase-2 inhibitor, such as 5-bromo-2-(4-fluorophenyl)-3-[4-(methylsulfonyl)phenyl]thiophene or diclofenac, is administered in a separate group.

In detail, male Wistar rats (5 animals per group, weighing approximately 200 g, supplied by Iffa Credo, France) are injected i.d. (intra-dermally) at the base of the tail with 0.1 ml of mineral oil containing 0.6 mg of lyophilised heat-killed Mycobacterium tuberculosis. The rats are treated with the test compound (3, 10 or 30 mg/kg p.o. once per day), or vehicle (water) from day 15 to day 22 (therapeutic dosing schedule). At the end of the experiment, the swelling of the tarsal joints is measured by means of a mico-calliper. Percentage inhibition of paw swelling is calculated by reference to vehicle treated arthritic animals (0 % inhibition) and vehicle treated normal animals (100% inhibition).

On the basis of these studies, a compound of formula I surprisingly is appropriate for the treatment of inflammatory (especially rheumatic or rheumatoid) diseases.

On the basis of their efficacy as inhibitors of VEGF-receptor tyrosine kinase activity, but also their efficacy as inhibitors of c-Abl protein-tyrosine kinase activity, the compounds of the formula I primarily inhibit the growth of blood vessels and are thus, for example, effective against a number of diseases associated with deregulated angiogenesis, especially diseases caused by ocular neovascularisation, especially retinopathies, such as diabetic retinopathy or age-related macular degeneration, psoriasis, haemangioblastoma, such as haemangioma, mesangial cell proliferative disorders, such as chronic or acute renal diseases, e; g. diabetic nephropathy, malignant nephrosclerosis, thrombotic microangio-pathy syndromes or transplant rejection, or especially inflammatory renal disease, such as glomerulonephritis, especially mesangioproliferative glomerulonephritis, haemolytic-uraemic syndrome, diabetic nephropathy, hypertensive nephrosclerosis, atheroma, arterial restenosis, autoimmune diseases, acute inflammation, fibrotic disorders (e.g. hepatic cirrhosis), diabetes, endometriosis, chronic asthma, arterial or post-transplantational atherosclerosis, neurodegenerative disorders and especially neoplastic diseases like leukaemias, especially acute lymphoblastic leukaemia, acute myeloid leukaemia and chronic myeloid leukaemia, and other “liquid tumours”, especially those expressing c-kit, KDR or fit-1, and solid tumours, especially breast cancer, cancer of the colon, lung cancer (especially small-cell lung cancer), cancer of the prostate or Kaposi's sarcoma. A compound of formula I (or an N-oxide thereof) inhibits the growth of tumours and is especially suited to preventing the metastatic spread of tumours and the growth of micrometastases.

A compound of formula I can be administered alone or in combination with one or more other therapeutic agents, possible combination therapy taking the form of fixed combinations or the administration of a compound of the invention and one or more other therapeutic agents being staggered or given independently of one another, or the combined administration of fixed combinations and one or more other therapeutic agents. A compound of formula I can besides or in addition be administered especially for tumor therapy in combination with chemotherapy, radiotherapy, immunotherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Therapeutic agents for possible combination are especially one or more antiproliferative, cytostatic or cytotoxic compounds, for example a chemotherapeutic agent or several agents selected from the group which includes, but is not limited to, an inhibitor of polyamine biosynthesis, an inhibitor of a protein kinase, especially of a serine/threonine protein kinase, such as protein kinase C, or of a tyrosine protein kinase, such as the EGF receptor tyrosine kinase or the PDGF receptor tyrosine kinase, e.g. STI571 (GLEEVEC®), a cytokine, a negative growth regulator, such as TGF-β or IFN-β, an aromatase inhibitor, e.g. letrozole or anastrozole, an inhibitor of the interaction of an SH2 domain with a phosphorylated protein, antiestrogens, topoisomerase I inhibitors, such as irinotecan, topoisomerase II inhibitors, microtubule active agents, e.g. paclitaxel, discodermolide or an epothilone, alkylating agents, antineoplastic antimetabolites, such as gemcitabine or capecitabine, platin compounds, such as carboplatin or cisplatin, anti-angiogenic compounds, gonadorelin agonists, anti-androgens, bisphosphonates, e.g. AREDIA® or ZOMETA®, and trastuzumab. The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference.

With the groups of preferred compounds of formula I and N-oxides thereof mentioned hereinafter, definitions of substituents from the general definitions mentioned hereinbefore may reasonably be used, for example, to replace more general definitions with more specific definitions or especially with definitions characterized as being preferred;

Furthermore, the invention relates to the use of a compound of formula 1, wherein the radicals and symbols have the meanings as defined above, or a N-oxide or a pharmaceutically acceptable salt thereof for the preparation of a pharmaceutical product for the treatment of retinopathy or age-related macula degeneration.

Furthermore, the invention relates to a method for the treatment of a neoplastic disease which responds to an inhibition of the VEGF-receptor tyrosine kinase activity, which comprises administering a compound of formula I or a N-oxide or a pharmaceutically acceptable salt thereof, wherein the radicals and symbols have the meanings as defined above, in a quantity effective against the said disease, to a warm-blooded animal requiring such treatment.

Furthermore, the invention relates to a method for the treatment of retinopathy or age-related macular degeneration, which comprises administering a compound of formula I or a N-oxide or a pharmaceutically acceptable salt thereof, wherein the radicals and symbols have the meanings as defined above, in a quantity effective against said diseases, to a warm-blooded animal requiring such treatment.

The invention relates in particular to a compound of formula I, wherein R represents H, halogen, alkynyl, alkenyl, alkyl, which in each case is unsubstituted or substituted by halogen; unsubstituted or substituted mono- or bicyclic aryl; unsubstituted or substituted mono- or bicyclic heteroaryl having 1 to 3 heteroatoms selected from O, N or S; unsubstituted or substituted heterocyclyl having at least one N atom; mono- or dialkyl amino, wherein the alkyl radical is unsubstituted or substituted by unsubstituted or substituted aryl, unsubstituted or substituted mono- or bicyclic heteroaryl having 1 to 3 heteroatoms selected from O, N or S or substituted by unsubstituted or substituted heterocyclyl having at least one N atom; unsubstituted or substituted heterocyclyl carbonyl alkyl amino, wherein the heterocyclyl radical comprises at least one N atom; R₁ represents H, halogen, C₂₋₇alkyl, C₂₋₇alkenyl, C₂₋₇alkynyl, alkoxy or a radical —O—(CH₂)ₙ-CF₃, wherein n is 0, 1, 2 or 3, R₂ is perfluoro alkyl, R₃ represents H or halogen, X represents hydroxy, alkoxy, alkyl thio, imino, alkyl imino, halogen, a radical of formula I′ 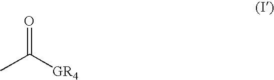
wherein G is CH₂ or NH and R₄ is hydrogen, alkyl or aryl, or a radical of formula I′ 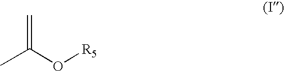
wherein R₅ is alkyl or aryl, Z is N or CH, and wherein the methylen group is attached to the pyridyl moiety at the carbon atom of the pyridyl moiety in 2-, 3-, 4- or 5-position, under the proviso that R cannot represent H, if Z is nitrogen, X is hydroxy or methoxy and wherein the methylen group is attached to the pyridyl moiety at the carbon atom of the pyridyl moiety in 3-position, and R₁ and R₃ cannot both represent H if Z is CH, R represents H, X is hydroxy, alkoxy or alkyl thio and wherein the methylen group is attached to the pyridyl moiety at the carbon atom of the pyridyl moiety in 3-position, or an N-oxide or a tautomer thereof, or a salt of such anthranilic acid amide, its N-oxide or its tautomer.

The invention further relates in particular to a compound of formula I, wherein R represents H, halogen, alkenyl, alkyl, pyridyl alkyl amino, morpholinyl alkyl amino, alkyl piperazinyl alkyl amino, alkyl piperazinyl carbonyl alkyl amino, phenyl alkyl amino, alkyl amino, thienyl, pyridyl, furanyl, thiazolyl, naphthyl or phenyl which is unsubstituted or substituted by trifluoromethyl, phenyl, alkanoyl or alkanoyl amino, R₁ represents H, halogen, C₂₋₇alkyl, C₂₋₇alkenyl, C₂₋₇alkynyl, alkoxy or a radical —O—(CH₂)ₙ-CF₃, wherein n is 0, 1, 2 or 3,

R₂ is perfluoro alkyl,

R₃ represents H or halogen,

X represents hydroxy, alkoxy, alkyl thio, imino, alkyl imino, halogen, a radical of formula I′ wherein G is CH₂ or NH and R₄ is hydrogen or alkyl, or a radical of formula I″ wherein R₅ is alkyl,

Z is N or CH, and

wherein the methylen group is attached to the pyridyl moiety at the carbon atom of the pyridyl moiety in 2-, 3-, 4- or 5-position, under the proviso that R cannot represent H, if Z is nitrogen, X is hydroxy or methoxy and wherein the methylen group is attached to the pyridyl moiety at the carbon atom of the pyridyl moiety in 3-position, and R₁ and R₃ cannot both represent H if Z is CH, R represents H, X is hydroxy, alkoxy or alkyl thio and wherein the methylen group is attached to the pyridyl moiety at the carbon atom of the pyridyl moiety in 3-position, to an N-oxide or a tautomer thereof, or to a salt of such anthranilic acid amide, its N-oxide or its tautomer.

More particularly, the invention relates to a compound of formula I, wherein R represents H, halogen, lower alkenyl, lower alkyl, pyridyl lower alkyl amino, morpholinyl lower alkyl amino, lower alkyl piperazinyl lower alkyl amino, lower alkyl piperazinyl carbonyl lower alkyl amino, phenyl lower alkyl amino, lower alkyl amino, thienyl, pyridyl, furanyl, thiazolyl, naphthyl or phenyl which is unsubstituted or substituted by trifluoromethyl, phenyl, lower alkanoyl or lower alkanoyl amino, R₁ represents H, halogen, C₂₋₇alkyl, C₂₋₇alkenyl, C₂₋₇alkynyl, lower alkoxy or a radical —O—(CH₂)ₙCF₃, wherein n is 0, 1, 2 or 3,

R₂ is trifluoromethyl,

R₃ represents H or halogen,

X represents hydroxy, lower alkoxy, lower alkyl thio, imino, lower alkyl imino, halogen, a radical of formula I′ wherein G is CH₂ or NH and R₄ is hydrogen or lower alkyl, or a radical of formula I″ wherein R₅ is lower alkyl,

Z is N or CH, and wherein the methylen group is attached to the pyridyl moiety at the carbon atom of the pyridyl moiety in 2-, 3-, 4- or 5-position, under the proviso that R cannot represent H, if Z is nitrogen, X Is hydroxy or methoxy and wherein the methylen group is attached to the pyridyl moiety at the carbon atom of the pyridyl moiety in 3-position, and R₁ and R₃ cannot both represent H if Z is CH, R represents H, X is hydroxy, lower alkoxy or lower alkyl thio and wherein the methylen group is attached to the pyridyl moiety at the carbon atom of the pyridyl moiety in 3-position, to an N-oxide or a tautomer thereof, or to a salt of such anthranilic acid amide, its N-oxide or its tautomer.

Preferred are compounds of formula I, wherein R represents H, halogen, lower alkenyl, lower alkyl, pyridyl lower alkyl amino, morpholinyl lower alkyl amino, lower alkyl piperazinyl lower alkyl amino, lower alkyl piperazinyl carbonyl lower alkyl amino, phenyl lower alkyl amino, lower alkyl amino, thienyl, pyridyl, furanyl, thiazolyl, naphthyl or phenyl which is unsubstituted or substituted by trifluoromethyl, phenyl, lower alkanoyl or lower alkanoyl amino,

R₁ represents H, halogen, C₂₋₇alkyl, C₂₋₇alkenyl, C₂₋₇alkynyl, lower alkoxy or a radical —O—(CH₂)ₙ-CF₃, wherein n is 0 or 1,

R₂ is trifluoromethyl,

R₃ represents H or halogen,

X represents hydroxy, lower alkoxy, halogen, a radical of formula I′ wherein R₄ is hydrogen or lower alkyl, or a radical of formula I″ wherein Rs is lower alkyl,

Z is N or CH, and wherein the methylen group is attached to the pyridyl moiety at the carbon atom of the pyridyl moiety in 3- or 4-position,

under the proviso that R cannot represent H, if Z is nitrogen, X Is hydroxy or methoxy and wherein the methylen group is attached to the pyridyl moiety at the carbon atom of the pyridyl moiety in 3-position, and R₁ and R₃ cannot both represent H if Z is CH, R represents H, X is hydroxy, lower alkoxy or lower alkyl thio and wherein the methylen group is attached to the pyridyl moiety at the carbon atom of the pyridyl moiety in 3-position, and their N-oxides and tautomers, and a salt of such compounds.

More preferred are compounds of formula I, wherein

R represents H, halogen, lower alkenyl, lower alkyl, pyridyl lower alkyl amino, morpholinyl lower alkyl amino, lower alkyl piperazinyl lower alkyl amino, lower alkyl piperazinyl carbonyl lower alkyl amino, phenyl lower alkyl amino, lower alkyl amino, thienyl, pyridyl, furanyl, thiazolyl, naphthyl or phenyl which is unsubstituted or substituted by trifluoromethyl, phenyl, lower alkanoyl or lower alkanoyl amino,

R₁ represents H, halogen, C₂₋₇alkyl, or C₂₋₇alkynyl,

R₂ Is trifluoromethyl,

R₃ represents H or halogen,

X represents hydroxy, lower alkoxy, halogen, a radical of formula I′ wherein R₄ is hydrogen or lower alkyl, or a radical of formula I″ wherein R₅ is lower alkyl,

Z is CH, and

wherein the methylen group is attached to the pyridyl moiety at the carbon atom of the pyridyl moiety in 3- or 4-position, under the proviso that R₁ and R₃ cannot both represent H in compounds of formula I wherein R represents H, X is hydroxy, lower alkoxy or lower alkyl thio and wherein the methylen group is attached to the pyridyl moiety at the carbon atom of the pyridyl moiety in 3-position.

In particular, preferred compounds of formula I are those in which R represents H, halogen, allyl, 3-methyl-buten-2-yl, propyl, ethylamino, pyridylethylamino, morpholinylethylamino, N-methyl-piperazinylpropylamino, N-methyl-piperazinylethylamino, N-methyl-piperazinylacetylamino, benzylamino, thienyl, pyridyl, furanyl, thiazolyl, naphthyl or phenyl which is unsubstituted or substituted by trifluoromethyl, phenyl, formyl or acetylamino,

R₁ represents H, halogen, propyl, propynyl,

R₂ is trifluoromethyl,

R₃ represents H or halogen,

X represents hydroxy, lower alkoxy, halogen, a radical of formula I′ wherein R₄ is hydrogen or lower alkyl, or a radical of formula I″ wherein R₅ Is lower alkyl,

Z is CH, and

wherein the methylen group is attached to the pyridyl moiety at the carbon atom of the pyridyl moiety in 3- or 4-position, under the proviso that R₁ and R₃ cannot both represent H in compounds of formula I wherein R represents H, X is hydroxy, lower alkoxy or lower alkyl thio and wherein the methylen group is attached to the pyridyl moiety at the carbon atom of the pyridyl moiety in 3-position.

Furthermore preferred are compounds of formula I, wherein R represents halogen, lower alkenyl, lower alkyl, pyridyl lower alkyl amino, morpholinyl lower alkyl amino, lower alkyl piperazinyl lower alkyl amino, lower alkyl piperazinyl carbonyl lower alkyl amino, phenyl lower alkyl amino, lower alkyl amino, thienyl, pyridyl, furanyl, thiazolyl, naphthyl or phenyl which is unsubstituted or substituted by trifluoromethyl, phenyl, lower alkanoyl or lower alkanoyl amino,

R₁ represents H,

R₂ is trifluoromethyl,

R₃ represents H,

X represents hydroxy or lower alkoxy,

Z is CH, and wherein the methylen group is attached to the pyridyl moiety at the carbon atom of the pyridyl moiety in 3- or 4-position.

More specifically, preference is given to the following compounds of formula I:
2-[[6-Methoxy-3-pyridinyl]methyl]amino-N-[4-bromo-3-(trifluoromethyl)phenyl]benzamide,
2-[[2-Bromo-4-pyridinyl]methyl]amino-N-[(3-trifluoromethyl)phenyI)benzamide,
2-[[6-Methoxy-4-pyridinyl]methyl]amino-N-[3-(trifluoromethyl)phenyl]benzamide,
2-[[6-Methoxy-3-pyridinyl]methyl]amino-N-[2-fluoro-3-(teifluoromethyl)phenyllbenzamide,
2-[[6-Methoxy-3-pyridinyi]methyl]amino-N-[4-chloro-3-(trifluoromethyi)phenyllbenzamide,
2-[[6-Methoxy-3-pyridinyl]methyl]amino-N-[4-(1 -propynyl)-3-(trifluoromethyl)phenyl]-benzamide,
2-[[6-Methoxy-3-pyridinyl]methyl]amino-/-[4-(1 -propyl)-3-(trifluoromethyl)phenyl]benzamide hydrochloride salt,
2-[[1,6-Dihydro-6-oxo-3-pyridinyl)methyl]amino]-N-[4-propynyl-3-(trifluoro-methyl)phenyl]benzamide,
2-[[(1,6-Dihydro-6-oxo-3-pyridinyl)methyllamino]-N-[4-propyl-3-(trifluoromethyl)-phenyl]benzamide,
2-[[(1,6-Dihydro-6-oxo-3-pyridinyl)methyl]aminol-N-[2-fluoro-3-(trifluoromethyl)-phenyl]benzamide,
2-[[(1,6-Dihydro-6-oxo-3-pyridinyl)methyl]aminol-N-[4-chloro-3-(trifluoromethyl)-phenyl]benzamide,
2-[[2-(1 -Ethoxyethenyl)-4-pyridinyl]methyl]amino-N[(3-trifluoromethyl)phenyl)benzamide,
2-[(2-Acetyl-4-pyridinyl)methyl]amino-N-[(3-trifluoromethyl)phenyl)benzamide,
2-[[(1,6-Dihydro-6-oxo-3-pyridinyl)methyl]amino]-N-[4-(2,2,2-trifluoroethoxy)-3-(trifluoromethyl)phenyl]benzamide,
2-[[(1,6-Dihydro-6-oxo-3-pyridinyl)methyl]aminol-N-[2-fluoro-4-(2,2,2-trifluoroethoxy)-3-(trifluoromethyl)phenyl]benzamide,
2-[[(1,6-Dihydro-6-oxo-3-pyridinyl)methyl]amino]-N-[4-(2,2,2-trifluoropropoxy)-3-(trifluoromethyl)phenyl]benzamide,
2-[[(1,6-Dihydro-6-oxo-3-pyridinyl)methyl]amino]-N-[4-trifluoromethoxy)-3-(trifluoromethyl)phenyl]benzamide,
2-[[(1,6-Dihydro-6-oxo-3-pyridinyl)methyl]amino]-N-[4-(2,2,2-trifluoroethoxy)-3-(trifluoromethyl)phenyl]nicotinamide,
2-[[6-Methoxy-3-pyridinyl]methyl]amino-N-[4-fluoro-3-(trifluoromethyl)phenyl]benzamide,
2-[[(1,6-Dihydro-6-oxo-3-pyridinyl)methyl]amino]-N-[4-fluoro-3-(trifluoromethyl)-phenyl]benzamide,
2-[(5-Bromo-6-methoxy-pyridin-3-ylmethyl)-amino]-N-(3-trifluoromethyl-phenyl)-benzamide,
2-[[(1,6-Dihydro-5-bromo-6-oxo-3-pyridinyl)methyl]amino]-N-[3-(trifluoromethyl)-phenyl]benzamide,
2-[(6-Methoxy-5-phenyl-pyridin-3-ylmethyl)-amino]-N-(3-trifluoromethyl-phenyl)-benzamide,
2-[(6-Oxo-5-phenyl-1,6-dihydro-pyridin-3-ylmethyl)-amino]-N-(3-trifluoromethyl-phenyl)-benzamide,
2-[(5-Allyl-6-methoxy-pyridin-3-ylmethyl)-amino]-N-(3-trifluoromethyl-phenyl)-benzamide,
2-[(5-ⁿPropyl-6-methoxy-pyridin-3-ylmethyl)-amino]-N-(3-trifluoromethyl-phenyl)-benzamide,
2-[(5-Allyl-6-oxo-1,6-dihydro-pyridin-3-ylmethyl)-amino]-N-(3-trifluoromethyl-phenyl)-benzamide,
2-[(5-ⁿ Propyl-6-oxo-1,6-dihydro-pyridin-3-ylmethyl)-amino]-N-(3-trifluoromethyl-phenyl)-benzamide,
2-[(5-Ethylamino-6-methoxy-pyridin-3-ylmethyl)-amino]-N-(3-trifiuoromethyl-phenyl)-benzamide,
2-[(5-Ethylamino-6-oxo-1,6-dihydro-pyridin-3-ylmethyl)-amino]-N-(3-trifluoromethyl-phenyl)-benzamide,
2-({5-[2-(4-Methyl-piperazin-1-yl)-ethylamino]-6-oxo-1,6-dihydro-pyridin-3-ylmethyl}-amino)-N-(3-trifluoromethyl-phenyl)-benzamide,
2-({6-Methoxy-5-[2-(4-methyl-piperazin-1-yl)-ethylamino]-pyridin-3-ylmethyl}-amino)-N-(3-trifluoromethyl-phenyl)-benzamide,
2-({5-[2-(4-Methyl-piperazin-1-yl)-ethylamino]-6-oxo-1,6-dihydro-pyridin-3-ylmethyl}-amino)-N-(3-trifluoromethyl-phenyl)-benzamide,
2-{[(1,6-Dihydro-6-oxo-3-pyridinyl)methyl]amino}-N -(4-methyl-3-trifluoromethyl-phenyl)benzamide,
2-{[(1,6-Dihydro-6-oxo-3-pyridinyl)methyl]amino}-N-[3-(4-ethyl-piperazin-1-ylmethyl)-5-trifluoromethyl-phenyl]benzamide,
2-{[(1,6-Dihydro-6-oxo-3-pyridinyl)methyl]amino}-N-[3-(azetidin-1-ylmethyl)-5-trfluoromethyl-phenyl]benzamide, phenyl]benzamide,
2-{[(1,6-Dihydro-6-oxo-3-pyridinyl)methyl]amino}-N-[4-(4-methyl-piperazin-1-ylmethyl)-3-trifluoromethyl-phenyl]benzamide,
2-{[(1,6-Dihydro-6-oxo-3-pyridinyl)methyl]amino}-N-4-[[2-(dimethylamino)ethyl]methylamino]-3-trifluoromethyl-phenyl]benzamide, and
2-{[(1,6-Dihydro-6-oxo-3-pyridinyl)methyl]amino}-N-5-(5-Methyl-1H-imidazol-1-yl)-3-trifluoromethyl-phenyl]benzamide,
or a tautomer thereof,
or a salt of such anthranilic acid amide or its tautomer.

Furthermore, special preference is given to the compounds of formula I, wherein R₁ and R₃ are H, R₂ is CF₃, Z is CH, X is OH or OMe, the methylen group is attached to the pyridyl moiety at the carbon atom of the pyridyl moiety in 3-position and
R is a radical selected from the following group:

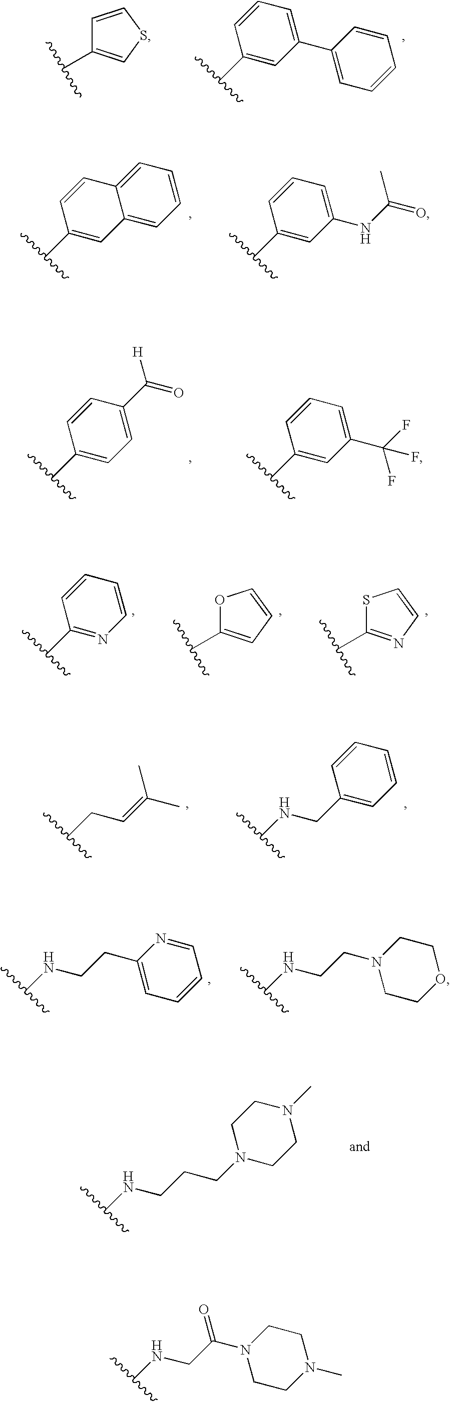

A compound of the invention may be prepared by processes that, though not applied hitherto for the new compounds of the present invention, are known per se, especially by a process characterized in that for the synthesis of a compound of the formula I wherein X represents lower alkoxy, lower alkylthio, lower alkylimino or halogen and the remaining symbols R₁, R₂, R₃ and Z are as defined for a compound of the formula I, compound of the formula II

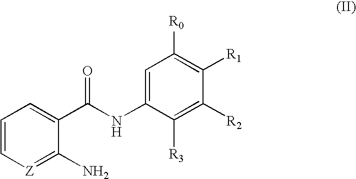

wherein R₀, R₁, R₂, R₃ and Z are as defined for a compound of the formula I, is reacted with a carbonyl compound of the formula III

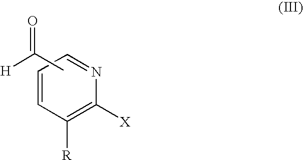

wherein X represents lower alkoxy, lower alkylthio, lower alkylimino or halogen and R has the meaning as provided above for a compound of formula I in the presence of a reducing agent,

wherein the starting compounds of formula II and III may also be present with functional groups in protected form if necessary and/or in the form of salts, provided a salt-forming group is present and the reaction in salt form is possible;

wherein any protecting groups in a protected derivative of a compound of the formula I are removed;

and, if so desired, an obtainable compound of formula I is converted into another compound of formula I or a N-oxide thereof, a free compound of formula I is converted into a salt, an obtainable salt of a compound of formula I is converted into the free compound or another salt, and/or a mixture of isomeric compounds of formula I is separated into the individual isomers.

Detailed description of the reductive alkylation:

In the more detailed description of the process below, R, R₀, R₁, R₂, R₃ and Z are as defined for compounds of formula I, unless otherwise indicated.

The carbonyl compound of the formula III may also be present in the form of reactive derivative; however, the free aldehyde or ketone is preferred.

Reactive derivatives of the compounds of formula III are, for example, corresponding bi-sulfite adducts or especially semiacetals, acetals, semiketals or ketals of compounds of formula III with alcohols, for example lower alkanols; or thioacetals or thioketals of compounds of formula III with mercaptans, for example lower alkanesulfides.

The reductive alkylation is preferably carried out with hydrogenation in the presence of a catalyst, especially a noble metal catalyst, such as platinum or especially palladium, which is preferably bonded to a carrier material, such as carbon, or a heavy metal catalyst, such as Raney nickel, at normal pressure or at pressures of from 0.1 to 10 MegaPascal (MPa), or with reduction by means of complex hydrides, such as borohydrides, especially alkali metal cyanoborohydrides, for example sodium cyanoborohydride, in the presence of a suitable acid, preferably relatively weak acids, such as lower alkanecarboxylic acids, especially acetic acid, or a sulfonic acid, such as p-toluenesulfonic acid; in customary solvents, for example alcohols, such as methanol or ethanol, or ethers, for example cyclic ethers, such as tetrahydrofuran, in the presence or absence of water.

Protecting groups

If one or more other functional groups, for example carboxy, hydroxy, amino, or mercapto, are or need to be protected in a compound of formulae II or III, because they should not take part in the reaction, these are such groups as are usually used In the synthesis of peptide compounds, and also of cephalosporins and penicillins, as well as nucleic acid derivatives and sugars.

The protecting groups may already be present in precursors and should protect the functional groups concerned against unwanted secondary reactions, such as acylations, etherifications, esterifications, oxidations, solvolysis, and similar reactions. It is a characteristic of protecting groups that they lend themselves readily, i.e. without undesired secondary reactions, to removal, typically by solvolysis, reduction, photolysis or also by enzyme activity, for example under conditions analogous to physiological conditions, and that they are not present in the end-products. The specialist knows, or can easily establish, which protecting groups are suitable with the reactions mentioned hereinabove and hereinafter.

The protection of such functional groups by such protecting groups, the protecting groups themselves, and their removal reactions are described for example in standard reference works, such as J. F. W. McOmie, “Protective Groups in Organic Chemistry”, Plenum Press, London and New York 1973, in T. W. Greene, “Protective Groups in Organic Synthesis”, Wiley, N.Y. 1981, in “The Peptides”; Volume 3 (editors: E. Gross and J. Melenhofer), Academic Press, London and New York 1981, in “Methoden der organischen Chemie” (Methods of organic chemistry), Houben Weyl, 4th edition, Volume 15/I, Georg Thieme Verlag, Stuttgart 1974, in H.-D. Jakubke and H. Jescheit, “Aminosäuren, Peptide, Proteine”(Amino acids, peptides, proteins), Verlag Chemie, Weinheim, Deerfield Beach, and Basel 1982, and in Jochen Lehmann, “Chemle der Kohlenhydrate: Monosaccharide und Derivate”(Chemistry of carbohydrates: monosaccharides and derivatives), Georg Thieme Verlag, Stuttgart 1974.

Additional process steps

Salts of a compound of formula I with a salt-forming group may be prepared in a manner known per se. Acid addition salts of compounds of formula I may thus be obtained by treatment with an acid or with a suitable anion exchange reagent. A salt with two acid molecules (for example a dihalogenide of a compound of formula I) may also be converted into a salt with one acid molecule per compound (for example a monohalogenide); this may be done by heating to a melt, or for example by heating as a solid under a high vacuum at elevated temperature, for example from 130 to 170° C., one molecule of the acid being expelled per molecule of a compound of formula I.

Salts can usually be converted to free compounds, e.g. by treating with suitable basic agents, for example with alkali metal carbonates, alkali metal hydrogencarbonates, or alkali metal hydroxides, typically potassium carbonate or sodium hydroxide.

An anthranilic acid amide of formula I wherein X represents lower alkoxy, lower alkylthio, lower alkylimino or halogen, obtained by reaction of the compounds of formula II and formula III, can be further reacted in accordance with the following processes (a) and (b) or both.

Process (a): An anthranilic acid amide of formula I wherein X represents lower alkoxy and the remaining symbols and radicals are as defined as for a compound of formula I is transferred into a compound of formula I wherein X is hydroxy, e.g., by treatment with trimethylsilyl iodide for about 20 to 35 hours at a temperature between 45° C. and 70° C. in a suitable solvent, e.g. a halogenated alkane, like chloroform, optionally followed by treatment with methanol.

Process (b): An anthranilic acid amide of formula I wherein X represents halogen, preferably bromide, and the remaining symbols and radicals are as defined as for a compound of formula I is transferred into a compound of formula I wherein X represents a radical of formula I″ by reaction with a stannane of formula VII,

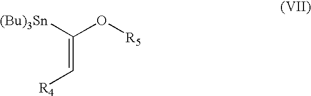

wherein R₄ is H or lower alkyl and R₅ represents lower alkyl, e.g. tributyl-(1-ethoxyethenyl)-stannane, in the presence of a suitable catalyst, preferably tetrakis(triphenylphosphine)-palladium (0), at a temperature between 90° C. and 150° C., preferably under an argon atmosphere between 12 and 48 hours in a suitable aromatic solvent like benzene, toluene or xylene.

The obtained compound of formula I wherein X represents a radical of formula I″ can be further transformed into a compound of formula I wherein X represents a radical of formula I′ by reacting the compound of formula I wherein X represents a radical of formula I″ solved in a suitable solvent, e.g. a mixture of an alcohol, like isopropanol, and tetrahydrofuran with a solution of hydrogen chloride in dioxan or tetrahydrofuran, for a duration between 1 and 12 hours at a temperature between 10° C. and 35° C.

An anthranilic acid amide of formula I wherein R₁ represents halogen, preferably bromide, can be further reacted in accordance with the following process (c).

Process (c): An anthranilic acid amide of formula I wherein R₁ represents halogen is solved in a suitable aromatic solvent like benzene, toluene or xylene and reacted with a stannane of formula VIII,

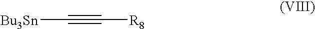

wherein R₈ is H or lower alkyl, in the presence of a suitable catalyst, preferably tetrakis-(triphenylphosphine)palladium (0), at a temperature between 90° C. and 150° C., preferably under an argon atmosphere, between 12 and 36 hours in a suitable aromatic solvent like benzene, toluene or xylene.

The obtained compound of formula I wherein R₁ represents the alkynyl radical —C≡C—R₈ can be transformed into a corresponding alkenyl or alkyl radicals by reduction reactions known in the art. For example, a compound of formula I wherein R₁ represents the alkynyl radical —C≡C—R₈ can be hydrogenated in methanol at atmospheric pressure over 5% platinum on carbon at a temperatur between 15° C. and 30° C., to provide a compound of formula I wherein R₁ represents C₂₋₇alkyl.

General process conditions

All process steps described here can be carried out under known reaction conditions, preferably under those specifically mentioned, in the absence of or usually in the presence of solvents or diluents, preferably such as are inert to the reagents used and able to dissolve these, in the absence or presence of catalysts, condensing agents or neutralisiing agents, for example ion exchangers, typically cation exchangers, for example in the H⁺form, depending on the type of reaction and/or reactants at reduced, normal, or elevated temperature, for example in the range from −100° C. to about 190° C., preferably from about −80° C. to about 150° C., for example at −80 to −60° C., at room temperature, at - 20 to 40° C. or at the boiling point of the solvent used, under atmospheric pressure or in a closed vessel, where appropriate under pressure, and/or in an inert atmosphere, for example under argon or nitrogen.

Salts may be present in all starting compounds and transients, if these contain salt-forming groups. Salts may also be present during the reaction of such compounds, provided the reaction is not thereby disturbed.

The solvents from which those can be selected which are suitable for the reaction in question include for example water, esters, typically lower alkyl-lower alkanoates, e.g diethyl acetate, ethers, typically aliphatic ethers, e.g. diethylether, or cyclic ethers, e.g. tetrahydrofuran, liquid aromatic hydrocarbons, typically benzene or toluene, alcohols, typically methanol, ethanol or 1- or 2-propanol, nitrites, typically acetonitrile, halogenated hydrocarbons, typically dichloromethane, acid amides, typically dimethylformamide, bases, typically heterocyclic nitrogen bases, e.g. pyridine, carboxylic acids, typically lower alkanecarboxylic acids, e.g. acetic acid, carboxylic acid anhydrides, typically lower alkane acid anhydrides, e.g. acetic anhydride, cyclic, linear, or branched hydrocarbons, typically cyclohexane, hexane, or isopentane, or mixtures of these solvents, e.g. aqueous solutions, unless otherwise stated in the description of the process. Such solvent mixtures may also be used in processing, for example through chromatography or distribution.

The compounds of formula I, including their salts, are also obtainable in the form of hydrates, or their crystals can include for example the solvent used for crystallization (present as solvates).

In the preferred embodiment, a compound of formula I is prepared according to or in analogy to the processes and process steps defined in the Examples.

The dosage of the active ingredient depends upon a variety of factors including type, species, age, weight, sex and medical condition of the patient; the severity of the condition to be treated; the route of administration; the renal and hepatic function of the patient; and the particular compound employed. A physician, clinician or veterinarian of ordinary skill can readily determine and prescribe the effective amount of the drug required to prevent, counter or arrest the progress of the condition. Optimal precision in achieving concentration of drug within the range that yields efficacy without toxicity requires a regimen based on the kinetics of the drug's availability to target sites. This involves a consideration of the distribution, equilibrium, and elimination of a drug.

The dose of a compound of the formula I or a pharmaceutically acceeptable salt thereof to be administered to warm-blooded animals, for example humans of approximately 70 kg body weight, is preferably from approximately 3 mg to approximately 5 g, more preferably from approximately 10 mg to approximately 1.5 g, most preferably from about 100 mg to about 1000 mg per person per day, divided preferably into 1 to 3 single doses which may, for example, be of the same size. Usually, children receive half of the adult dose.

The invention relates also to pharmaceutical compositions comprising an effective amount, especially an amount effective in the treatment of one of the above-mentioned disorders, of an anthranilic acid amide of formula I or an N-oxide or a tautomer thereof together with pharmaceutically acceptable carriers that are suitable for topical, enteral, for example oral or rectal, or parenteral administration and that may be inorganic or organic, solid or liquid. There are used for oral administration especially tablets or gelatin capsules that comprise the active ingredient together with diluents, for example lactose, dextrose, mannitol, and/or glycerol, and/or lubricants and/or polyethylene glycol. Tablets may also comprise binders, for example magnesium aluminum silicate, starches, such as corn, wheat or rice starch, gelatin, methylcellulose, sodium carboxymethylcellulose and/or polyvinylpyrrolidone, and, if desired, disintegrators, for example starches, agar, alginic acid or a salt thereof, such as sodium alginate, and/or effervescent mixtures, or adsorbents, dyes, flavorings and sweeteners. It is also possible to use the pharmacologically active compounds of the present invention in the form of parenterally administrable compositions or in the form of infusion solutions. The pharmaceutical compositions may be sterilized and/or may comprise excipients, for example preservatives, stabilisers, wetting agents and/or emulsifiers, solubilisers, salts for regulating the osmotic pressure and/or buffers. The present pharmaceutical compositions, which may, if desired, comprise other pharmacologically active substances are prepared in a manner known per se, for example by means of conventional mixing, granulating, confectioning, dissolving or lyophilising processes, and comprise approximately from 1% to 95%, especially from approximately 1% to approximately 20%, active ingredient(s).

Furthermore, the invention relates to a pharmaceutical composition for treatment of tumours in warm-blooded animals, including humans, comprising an antitumourally effective dose of a compound of the formula I as described above or a pharmaceutically acceptable salt of such a compound together with a pharmaceutical carrier.

Additionally, the present invention provides an anthranilic acid amide of formula I or an N-oxide or a tautomer thereof, or a pharmaceutically acceptable salt of such a compound, for use in a method for the treatment of the human or animal body.

The present invention also relates to the use of an anthranilic acid amide of formula I or an N-oxide or a tautomer thereof, or a pharmaceutically acceptable salt of such a compound, for the preparation of a pharmaceutical product for the treatment of a neoplastic disease, retinopathy or age-related macula degeneration.

In addition to this, the present invention teaches a method for the treatment of a neoplastic disease which responds to an inhibition of the VEGF-receptor tyrosine kinase activity, which comprises administering an anthranilic acid amide of formula I or a N-oxide or a tautomer thereof, or a pharmaceutically acceptable salt of such anthranilic acid amide, its N-oxide or its tautomer, in a quantity effective against said disease, to a warm-blooded animal requiring such treatment.

Starting materials

New starting materials and/or intermediates, as well as processes for the preparation thereof, are likewise the subject of this invention. In the preferred embodiment, such starting materials are used and reaction conditions so selected as to enable the preferred compounds to be obtained.

Starting materials of the formula III, IV and V are known, commercially available, or can be synthesized in analogy to or according to methods that are known in the art.

For example, a compound of the formula II can be prepared by the reduction of a nitro compound of the formula IV, 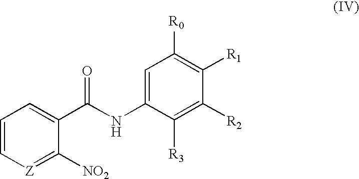
wherein R₀ to R₃ and Z have the meanings as given under formula I.

The reduction preferably takes place in the presence of a suitable reducing agent, such as tin(II) chloride or hydrogen in the presence of an appropriate catalyst, such as Raney nickel (then preferably the hydrogen is used under pressure, e.g. between 2 and 20 bar) or PtO₂, in an appropriate solvent, e.g. an alcohol, such as methanol. The reaction temperature is preferably between 0 and 80° C., especially 15 to 30° C.

A nitro compound of the formula IV is accessible by reaction of an activated acid derivative of the formula VI, 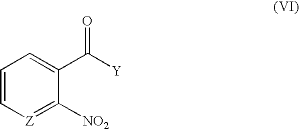
wherein Z is as defined above and Y is halogen or another suitable leaving group, is reacted with an amine of the formula V, 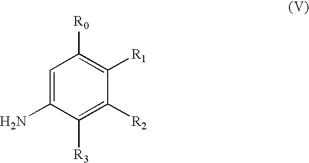
wherein R₁ to R₃ are as defined under formula I, e.g. in the presence of a coupling agent, such as dicyclohexylcarbodiimide, at a temperature between 0° C. and 50° C., preferably at room temperature.

Altematively, compounds of formula I can be obtained by a process characterized in that for the synthesis of a compound of the formula I wherein the symbols X, R₁, R₂, R₃ and Z are as defined for a compound of the formula I, a compound of the formula II 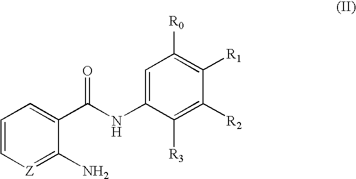
wherein R₁, R₂, R₃ and Z are as defined for a compound of the formula I, is reacted in a first step in an acetalisation reaction with a carbonyl compound of the formula III 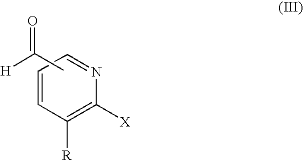
wherein X and R have the meanings as provided above for a compound of formula I, in a suitable solvent, e.g. benzene or toluene, in the presence of an acid, e.g. p-toluenesulfonic acid or, preferably, camphor-10-sulfonic acid, and under removal of the resulting water, e.g. by use of an equipment to remove the obtained water, like a water separator, or in the presence of a chemical compound reacting with the obtained water, wherein the starting compounds of formula II and III may also be present with functional groups in protected form if necessary and/or in the form of salts, provided a salt-forming group is present and the reaction in salt form is possible, providing an N,N-acetal of formula IX 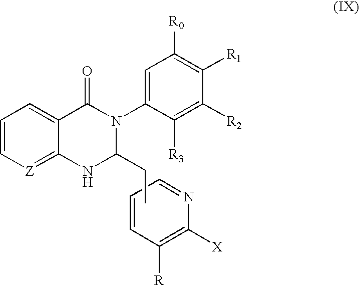
wherein X, R, R₀, R₁, R₂, R₃ and Z are as defined for a compound of the formula I.

In a second step, the N,N-acetal of formula IX is openend by reaction with a reducing agent, e.g. by reaction triethylsilane in the presence of trifluoroacetic acid for about 0.5 to 2 hours, e.g. 1 hour, at a temperature between −5° C. and +5° C., e.g. 0° C., in a suitable solvent, e.g. a lower alkane which is di- or trisubstituted by chlorine, providing a compound of formula I wherein X, R₁, R₂, R₃ and Z are as defined for a compound of the formula I above.

All remaining starting materials of are known, capable of being prepared according to known processes, or commercially obtainable; in particular, they can be prepared using processes as described in the Examples.

Abbreviations:
DMF dimethyltormamide
EtOAc ethyl acetate
Me methyl
m.p. melting point
MS mass spectra
RT room temperature
Tlc thin layer chromatogramme

The following Examples serve to illustrate the invention without limiting the invention in its scope.

Temperatures are measured in degrees celsius (°C.). Unless otherwise indicated, the reactions take place at room temperature (RT).

### EXAMPLES

### Example 1

### 2-[[6-Methoxy-3-pyridinyl]methyl]amino-N-[4-bromo-3-(trifluoromethyl)-phenyl]benzamide

Sodium cyanoborohydride (8.80 g of 95%, 133 mMol) is added in portions over 30 minutes to a stirred mixture of acetic acid (3.8 mL), 6-methoxy-3-pyridinecarboxaldehyde (Fluka, Buchs, Switzerland; 7.80 g, 57 mMol) and 2-amino-N-(4-bromo-3-trifluoromethylphenyl)-benzamide (step 1.2; 13.65 g, 38 mMol) in methanol (380 mL) at 25° C. The mixture is stirred for 16 hours. The solvent is evaporated under reduced pressure to give a residue which is treated with a saturated aqueous solution of sodium hydrogen carbonate (500 mL) and extracted with dichloromethane (3×150 mL). The combined extracts are dried (Na₂SO₄), filtered and the solvent is evaporated under reduced pressure to yield the crude product that is purified by column chromatography on silica gel, eluent 5% ethyl acetate in dichloromethane and recrystallised from diethylether-hexane to give the title compound as a beige crystalline solid, m.p. 101-103° C.

### Step 1.1

### 2-Nitro-N-(4-bromo-3-trifluoromethylphenyl)benzamide

A solution of 3-amino-6-bromobenzotrifluoride (Fluka, Buchs, Switzerland; 24.0 g, 100 mMol) in ethyl acetate (240 mL) is added to a stirred aqueous solution of sodium hydroxide (110 mL of 1 M) at room temperature. This stirred solution is then treated dropwise over 30 minutes with a solution of 2-nitrobenzoyl chloride (Fluka, Buchs, Switzerland; 14.5 mL, 110 mMol) in ethyl acetate (150 mL). The resulting mixture is then stirred for 30 min at ambient temperature. The mixture is extracted with ethyl acetate (3×100 mL) and the combined extracts are sequentially washed with hydrochloric acid (2×100 mL of 2 M), water (2×100 mL), saturated aqueous sodium hydrogen carbonate solution (2×100 mL) and saturated aqueous sodium chloride (1×100 mL), dried (MgSO₄), filtered and the solvent is evaporated under reduced pressure to yield the crude product which is purified by recrystallisation from ethyl acetate-hexane to give the title compound as a beige crystalline solid, m.p. 157 -158° C.

### Step 1.2

### 2-Amino-N-(4-bromo-3-trifluoromethylphenyl)benzamide

A solution of 2-nitro-N-(4-bromo-3-trifluoromethylphenyl)benzamide (intermediate 1a; 32 g, 82 mMol) in methanol (1000 mL) is hydrogenated at atmospheric pressure over Raney nickel (6 g) at 21° C. The calculated amount of hydrogen is taken up after 7 hours. The mixture is filtered and the solvent is evaporated under reduced pressure to yield the crude product which is purified by recrystallisation from diethylether-hexane to give the title compound as a colourless crystalline solid, m.p. 142-144° C.

### Example 2

### 2-[[2-Bromo-4-pyridinyl]methyl]amino-N-[(3-trifluoromethyl]phenyl)benzamide

The title compound is prepared by a method analogous to that described in Example 1 by utilising the intermediate from step 2.2 and 2-bromo-4-pyridinecarboxaldehyde (prepared according to the method described in EP 282077); m.p. 113-114° C.

### Step 2.1

### 2-Nitro-N-[3-(trifluoromethyl)phenyl]benzamide

The title compound is prepared analogously to step 1.1 by utilising 3-(trifluoromethyl)-benzenamine (Aldrich, Buchs, Switzerland); m.p. 134-135° C.

### Step 2.2

### 2-Amino-N-[(3-trifluoromethyl)phenyl)benzamide

The title compound is prepared analogously to step 1.2 by utilising 2-nitro-N-[(3-trifluoro-methyl)phenyl)benzamide (step 2.1); m.p. 132-133° C.

### Example 3

### 2[[6-Methoxy-4-pyridinyl]methyl]amino-N-[3-(trifluoromethyl)phenyllbenzamide

The title compound is prepared by a method analogous to that described in Example 1 by utilising the intermediate from step 2.2 and 6-methoxy-4-pyridinecarboxaldehyde (prepared according to the method described by T. H. Brown et al. in Eur. J. Med. Chem. 1993; 28:601-8); m.p. 113-114° C.

### Example 4

### 2[[6-Methoxy-3-pyridinyl]methyl]amino-N-[2-fluoro-3-(trifluoromethyl)phenyl-benzamide

The title compound is prepared by a method analogous to that described in Example 1 by utilising the intermediate from step 4.2 and 6-methoxy-3-pyridinecarboxaldehyde (Aldrich, Buchs, Switzerland); m.p. 123-125° C.

### Step 4.1

### 2-Nitro-N-[2-fluoro-3-(trifluoromethyl)phenyl]benzamide

The title compound is prepared analogously to step 1.1 by utilising 2-fluoro-3-(trifluoromethyl)-benzenamine (Aldrich, Buchs, Switzerland); m.p. 156-157° C.

### Step 4.2

### 2-Amino-N-[2-fluoro-(3-trifluoromethyl)phenyl)benzamide

The title compound is prepared analogously to step 1.2 by utilising 2-nitro-N-[2-fluoro-(3-trifluoromethyl)phenyl)benzamide (step 4.1); m.p. 128-129° C.

### Example 5

### 2-[[6-Methoxy-3-pyridinyl]methyl]amino-N-[4-chloro-3-(trifluoromethyl)-phenyl]benzamide

The title compound is prepared by a method analogous to that described in Example 1 by utlising the intermediate from step 5.2 and 6-methoxy-3-pyridinecarboxaldehyde (Aldrich, Buchs, Switzerland); m.p. 108-110° C.

### Step 5.1

### 2-Nitro-N-[4chloro-3-(trifluoromethyl)phenyl]benzamide

The title compound is prepared analogously to step 1.1 by utilising 4-chloro-3-(trifluoromethyl)benzenamine (Fluka, Buchs, Switzerland); m.p. 165-169° C.

### Step 5.2

### 2-Amino-N-[4-chloro-(3-trifluoromethyl)phenyl)benzamide

The title compound is prepared analogously to step 1.2 by utilising 2-nitro-N-[4-chloro-3-(trifluoromethyl)phenyl]benzamide (step 5.1); m.p. 148-150° C.

### Example 6

### 2-[[6-Methoxy-3-pyridinyl]methyl]amino-N-[4-(1-propynyl)-3-(trifluoromethyl)phenyl]benzamide

A stirred solution of 2-[[6-methoxy-3-pyridinyl]methyl]amino-N-[4-bromo-3-(trifluoromethyl)-phenyl]benzamide (Example 1; 3.98 g, 8.3 mMol) in dry toluene (200 mL) is purged with argon for 20 minutes at 250° C. Tributyl-1-propynylstannane (4.1 g of 80%, 9.96 mMol) and tetrakis(triphenylphosphine)palladium (0) (260 mg) are then added and the resulting mixture is heated at 100° C for 17 hours under an argon atmosphere. The mixture is then cooled, treated with an aqueous solution of sodium hydroxide (85 mL of 0.1 M) and purged with air for 2 hours. The resulting mixture is extracted with ethyl acetate (3×100 mL). The organic phase is sequentially washed with water (2×40 mL) and saturated aqueous sodium chloride (1×40 mL), dried (Na₂SO₄), filtered and the solvent is evaporated under reduced pressure to yield the crude product which is purified by column chromatography on silica gel, eluent 33% ethyl acetate in hexane and recrystallised from diethylether-hexane to give the title compound as a pale-yellow crystalline solid; m.p. 123-124° C.

### Example 7

### 2-[[6-Methoxy-3-pyridinyl[methyl[amino-N-[4-(1-propvl)-3-(trifluoromethyl)-phenyl]benzamide hydrochloride salt

A solution of 2-[[6-methoxy-3-pyridinyl]methyl]amino-N-[4-(1 -propynyl)-3-(trifluoromethyl)-phenyl]benzamide (Example 6; 1.85 g, 4.20 mMol) in methanol (100 mL) is hydrogenated at atmospheric pressure over 5% platinum on carbon (0.4 g) at 22° C. The calculated amount of hydrogen is taken up after 19 hours. The mixture is filtered and the solvent is evaporated under reduced pressure to yield the crude product which dissolved in ethanol, acidified with a solution of hydrogen chloride in ethyl acetate (0.9 M) and diluted with diethylether. The resulting precipitate is filtered off, dried and purified by recrystallisation from diethylether-ethanol to give the title compound as a yellow crystalline solid; m.p. 104-120° C.

### Example 8

### 2-[[(1,6-Dihydro-6-oxo-3-pyridinyl)methyl]amino]-N-[4-propnyl-3-(trifluoromethyl)phenyl]benzamide

A mixture of 2-[[6-methoxy-3-pyridinyl]methyl]amino-N-[4-(1-propynyl)-3-(trifluoromethyl)-phenyl]benzamide (Example 6; 1.10 g, 2.5 mMol) and trimethylsilyl iodide (Fluka, Buchs, Switzerland; 1.0 mL, 7.5 mMol) in chloroform (30 mL) is stirred at 60° C. for 16 hours under an argon atmosphere. The cooled mixture is then treated with methanol (2 mL) and stirred at room temperature for 10 minutes. The solvent is evaporated under reduced pressure and the residue is treated with an aqueous solution of ammonia (100 mL of 5%) and extracted with ethyl acetate (3×100 mL). The combined extracts are washed with saturated aqueous sodium chloride (50 mL), dried (MgSO₄), filtered and the solvent is evaporated under reduced pressure to yield the crude product which is purified by column chromatography on silica gel, eluent ethyl acetate and recrystallised from hot ethyl acetate-hexane to give the title compound as a colourless crystalline solid; m.p. 208-212° C.

### Example 9

### 2-[[(1,6-Dihydro-6-oxo-3-pyridlnyl)methyl]amino]-N-[4-propyl-3-(trifluoromethyl)-phenyl]benzamide

The title compound is prepared by a method analogous to that described in Example 8 by utilising 2-[[6-methoxy-3-pyridinyl]methyl]amino-N-[4-(1-propyl)-3-(trifluoromethyl)phenyl]-benzamide (Example 7); m.p. 195-199° C.

### Example 10

### 2-[[(1,6-Dihydro-6-oxo-3-pyridinyl)methyl]amino]-N-[2-fluoro-3-(trifluoromethvl)phenyl]benzamide

The title compound is prepared by a method analogous to that described in Example 8 by utilising 2-[[6-methoxy-3-pyridinyl]methyl]amino-N-[4-chloro-3-(trifluoromethyl)phenyl]-benzamide (Example 4); m.p. 185-186° C.

### Example 11

### 2-[[(1,6-Dihydro-6-oxo-3-pyridinyl)methyl]amino]-N-[4-chloro-3-(trifluoromethyl)phenyl]benzamide

The title compound is prepared by a method analogous to that described in Example 8 by utilising 2-[[6-methoxy-3-pyridinyl]methyl]amino-N-[4-chloro-3-(trifluoromethyl)phenyl]-benzamide (Example 5); m.p. 231° C.

### Example 12

### 2-[[2-(1-Ethoxyethenyl)-4-pyridinyl]methyl]amino-N-[(3-trifluoromethyl)phenyl)-benzamide

A stirred solution of 2-[[2-bromo-4-pyridinyl]methyl]amino-N-[(3-trifluoromethyl)phenyl)benzamide (Example 2; 0.38 g, 0.84 mMol) in dry toluene (25 mL) is purged with argon for 20 minutes at 25° C. Tributyl-(1-ethoxyethenyl)stannane (Fluka, Buchs, Switzerland; 343 mg, 0.92 mMol) and tetrakis(triphenylphosphine)palladium (0) (97 mg, 0.084 mMol) are added and the resulting mixture is heated at 125° C. for 38 hours under an argon atmosphere. The mixture is then cooled, diluted with toluene (50 mL) and washed with saturated aqueous ammonium chloride (2×40 mL). The toluene solution is dried (MgSO₄), filtered and the solvent is evaporated under reduced pressure to yield the crude product which is purified by column chromatography on silica gel, eluent 33% ethyl acetate in hexane and recrystallised from diethylether-hexane to give the title compound as a pale-yellow crystalline solid, m.p. 155-156° C.

### Example 13

### 2-[(2-Acetyl-4-pyridinyl)methyl]amino-Nr(3-trifluoromethyl)phenyl)benzamide

A stirred solution of 2-[[2-(1 -ethoxyethenyl)-4-pyridinyl]methyl]amino-N-[(3-trifluoromethyl)-phenyl)benzamide (Example 12; 0.18 g, 0.4 mMol) in a mixture of isopropanol (3.6 mL) and tetrahydrofuran (1.8 mL) is treated with a solution of hydrogen chloride in dioxan (0.5 mL of 4 M) and the resulting mixture is stirred at 25° C. for 6 hours under an argon atmosphere. The mixture is then treated with saturated aqueous ammonium chloride (40 mL), basified with aqueous ammonia (to pH 9 with 25%) and extracted with ethyl acetate (3×50 mL). The combined extracts are dried (MgSO₄), filtered and the solvent is evaporated under reduced pressure to yield the crude product which is purified by column chromatography on silica gel, eluent 33% ethyl acetate in hexane and recrystallised from diethylether-hexane to give the title compound as a colourless crystalline solid, m.p. 138-139° C.

### Example 14

The following compounds can be prepared in analogy to Example 8.
(a) 2-[[(1,6-Dihydro-6-oxo-3-pyridinyl)methyl]amino]-N-[4-(2,2,2-trifluoroethoxy)-3-(trifluoromethyl)phenyl]benzamide,
(b) 2-[[(1,6-Dihydro-6-oxo-3-pyridinyl)methyl]amino]-N-[2-fluoro4-(2,2,2-trifluoroethoxy)-3-(trifluoromethyl)phenyl]benzamide,
(c) 2-[[(1,6-Dihydro-6-oxo-3-pyridinyl)methyl]amino]-N-[4-(3,3,3-trifluoropropoxy)-3-(trifluoromethyl)phenyl]benzamide,
(d) 2-[[(1,6-Dihydro-6-oxo-3-pyridinyl)methyl]amino]-N-[4-trifluoromethoxy)-3-(trifluoromethyl)phenyl]benzamide,
(e) 2-[[(1,6-Dihydro-6-oxo-3-pyridinyl)methy(]amino]-N-[4-(2,2,2-trifluoroethoxy)-3-(trifluoromethyl)phenyl]nicotinamide.

### Example 15

### 2-[(6-Methoxy-3-pyridinyl]methyl]amino-N-[4-fluoro-3-(trifluoromethyl)phenyl]-benzamide

Under N₂-atmosphere, 6.4 g (15.3 mMol) rac. 3-(4-fluoro-3-trifluoromethyl-phenyl)-2-(6-methoxy-pyridin-3-yl)-2,3-dihydro-1 H-quinazolin-4-one are suspended in 30 ml of ice-cooled 1,2-dichloroethane. Then 3.73 ml (23 mMol) of triethylsilane are added during 5 min via syringe, followed by 7.4 ml (96.6 mMol) of trifluoroacetic acid. The resulting yellow solution is stirred for 1 h at 0° C. and 15 h at RT and then poored into ice-cold NaHCO₃ solution and 200 ml of dichloromethane. After 30 min stirring, the aqueous layer is separated off and extracted 3 times with dichloromethane. The organic phases are washed with water and brine, dried (Na₂SO₄) and concentrated in vacuuo. Column chromatography (SiO₂; crude product dissolved in CH₂Cl₂; eluted with hexane/EtOAc 2:1) and crystallization from CH₂Cl₂/hexane gives the title compound; m.p. 114-115° C.

### Step 15.1

### rac. 3-(4-Fluoro-3-trifluoromethyl-phenyl)-2-(6-methoxy-pyridin-3-yl)-2,3-dihydro-1H-quinazolin-4-one

To a suspension under N₂-atmosphere of 7.0 g (23.5 mMol) of 2-amino-N-[4-fluoro-3-(trifluoromethyl)phenyl]benzamide (preparation see WO 00/27820; intermediate 2h) in 65 ml of toluene, 3.22 g (23.5 mMol) of 6-methoxy-3-pyridinecarboxaldehyde and 10 mg of camphor-10-sulfonic acid are added. Then ≈30 ml of toluene are distilled off and replaced by fresh toluene. The resulting yellow solution is stirred for 5 h at 110° C. After cooling to RT, the reaction mixture is filtered and the filtrate concentrated. Column chromatography (SiO₂; CH₂Cl₂/MeOH 99:1→197:3) and crystallization from CH₂Cl₂/hexane gives the title compound; m.p. 167-168° C.

### Example 16

### 2-[[(1,6-Dihydro-6-oxo-3-pyidinyl)methyl]amino]-N-fluoro-3-(trifluoromethyl)-phenyl]benzamide

Under N₂-atmosphere, 2.02 g (5.00 mMol) rac. 3-(4-fluoro-3-trifluoromethyl-phenyl)-2-(6-oxo-1,6-dihydro-pyridin-3-yl)-2,3-dihydro-1H-quinazolin-4-one are suspended in 10 ml of ice-cooled 1,2-dichloroethane. Then 1.20 ml (7.5 mMol) bf triethylsilane are added during 2 min via syringe, followed after 5 min by 2.4 ml (31.5 mMol) of trifluoroacetic acid. The resulting yellow solution is stirred for 0.2 h at 0° C. and 6 h at RT and then poored into a mixture of diluted ice-cold NaHCO₃ solution and dichloromethane. The aqueous layer is separated off and extracted 3 times with dichloromethane. The organic phases are washed with water and brine, dried (Na₂SO₄) and concentrated in vacuuo. Column chromatography (SiO₂, EtOAc/hexane 9:1; crude product dissolved in EtOAc/MeOH 19:1; eluted with EtOAc/MeOH 19:1) and crystallization from CH₂Cl₂/hexane gives the title compound; m.p. 204-205° C.

### Step 16.1

### 6-Oxo-1,6-dihydro-pyridine-3-carbaldehyde

To a solution of 5.4 g (39.4 mMol) of 6-methoxy-3-pyridinecarboxaldehyde in 50 ml of dichloromethane, 5.4 ml (39 mMol) of Me₃Sil are added. This mixture is stirred for 1 h at RT and 1.5 h at 60° C. Then 6.4 ml of methanol are added to the reaction mixture at RT. After 10 min, the suspension was diluted by addition of more methanol. After adding 30 g of SiO₂, the mixture is concentrated and the resulting powder put on top of a chromatography column (CH₂Cl₂/acetone 7:3). Eluation with CH₂Cl₂/acetone 7:3→1:1→1:3 and crystallization from CH₂Cl₂/Et₂O gives the title compound; m.p. 222-224° C.

### Step 16.2:

### rac. 3-(4-Fluoro-3-trifluoromethyl-phenyl)-2-(6-oxo-1,6-dihydro-pyridin-3-yl)-2,3-dihydro-1 H-quinazolin-4-one

To a suspension under N₂-atmosphere of 2.98 g (10.0 mMol) of 2-amino-N-[4-fluoro-3-(trifluoromethyl)phenyl]benzamide (preparation see WO 00/27820; intermediate 2h) in 70 ml of toluene, 1.23 g (10.0 mMol) of 6-oxo-1,6-dihydro-pyridine-3-carbaldehyde and 10 mg of camphor-10-sulfonic acid are added. Then the mixture is heated up to the boiling temperature for 2 h. The condensate is passed through a Soxiet equipment containing molecular sieves (4 Å). After cooling to RT, the reaction mixture is filtered and the residue washed with diethyl ether, yielding the title compound; m.p. 260-261° C.

### Example 17

### 2-[(5-Bromo-6-methoxy-pyridin-3-ylmethyl)-amino]-N-(3-trifluoromethyl-phenyl)-benzamide

Under N₂-atmosphere, 68.0 g (142 mMol) rac. 3-(3-trifluoromethyl-phenyl)-2-(5-bromo-6-methoxy-pyridin-3-yl)-2,3-dihydro-1H-quinazolin-4-one are suspended in 280 ml of ice-cooled 1,2-dichloroethane. Then 34.8 ml (218 mMol) of triethylsilane are added dropwise during 5 min, followed by 68.7 ml (897 mMol) of trifluoroacetic acid (30 min). The reaction mixture is stirred for 1 h at 0° C. and 24 h at RT and then poored into a mixture of a diluted ice-cold NaHCO₃ solution and dichloromethane. After 30 min stirring, the aqueous layer is separated off and extracted 3 times with dichloromethane. The organic phases are washed with water and brine, dried (Na₂SO₄) and concentrated in vacuuo. Crystallizaton from diethyl ether/hexane gives the title compound; m.p. 110-111° C. More product can be obtained by column chromatography (SiO₂; CH₂Cl₂) and crystallization from CH₂Cl₂/hexane; m.p. 111-112° C.

### Step 17.1

### 5-Bromo-6-methoxy-3-pyridinecarboxaldehyde

(See Eur. J. Med. Chem.-Chim. Ther. 1977, 12, 531) 54.8 g (400 mMol) 6-methoxy-3-pyridinecarbaldehyde are dissolved in 180 ml of acetic acid. 63.8 g (778 mMol) sodium acetate are added portionwise (slightly exothermic). Then a solution of 30 ml (582 mMol) of bromine in 120 ml of acetic acid is added dropwise during 30 min. The mixture is stirred for 5 h at 90° C., then cooled to RT and concentrated partially in vaccuo. The residue is diluted with icewater, neutralized to pH 7.5 with 4 N NaOH and extracted with 4 portions of EtOAc. The organic layers are washed twice with water and brine, dried (Na₂SO₄) and concentrated in vacuuo. Column chromatography (SiO₂; CH₂Cl₂) of the resulting oil and crystallization from CH₂Cl₂/hexane gives the title compound; mp: 94-95° C.

### Step 17.2

### rac. 3-(3-Trifluoromethyl-phenyl)-2-(5-bromo-6-methoxy-pyridin-3-yl)-2,3-dihydro-1 H-quinazolin-4-one

To a suspension under N₂-atmosphere of 52.2 g (186 mMol) of 2-amino-N-[3-(trifluoromethyl)phenyl]benzamide (preparation see WO 00/27820; intermediate 2 m) in 470 ml of toluene, 40.3 g (186 mMol) of 5-bromo-6-methoxy-3-pyridinecarboxaldehyde and 111 mg of camphor-10-sulfonic acid are added. Then the mixture is heated up to the boiling temperature of toluene for 5 h on a water separation equipment. The resulting solution is concentrated in vacuuo. Crystallization from diethyl ether gives the title compound. More product can be obtained by column chromatography (SiO₂; CH₂Cl₂ →CH₂Cl₂/MeOH 99:1). Crystallization from CH₂Cl₂/hexane gives the title compound; m.p. 192-193° C.

### Example 18

### 2-[F(1.6-Dihydro-5-bromo-6-oxo-3-pyridinyl)methyl]amino]-N-[3-(trifluoromethyl)phenyl]benzamide

The title compound is prepared by a method analogous to that described in Example 8 by utilising 2-[(5-bromo-6-methoxy-pyridin-3-ylmethyl)-amino]-N-(3-trifluoromethyl-phenyl)-benzamide; m.p. 212-214° C.

### Example 19

### 2-(6-Methoxy-5-phenyl-pyridin-3-ylmethyl)-amino]-N-(3-trfluoromethyl-phenyl)-benzamide

To a solution of 240 mg (0.50 mMol) of 2-[(5-bromo-6-methoxy-pyridin-3-ylmethyl)-amino]-N-(3-trifluoromethyl-phenyl)-benzamide in 8 ml of DMF under N₂-atmosphere, 69 mg (97%; 0.55 mMol) of phenylboronic acid, 5.6 mg (0.025 mMol) Pd(OAc)₂, 15.2 mg (0.05 mMol) o-tolyl-phosphine and 1.3 ml of a 1 M solution of K₂CO₃ in water are added. The mixture is stirred for 1 h at 100° C., coolded to RT and diluted with water and EtOAc. The aqueous layer is separated off and extracted twice with EtOAc. The organic phases are washed with water and brine, dried (Na₂SO₄) and concentrated. Column chromatography (SiO₂; hexane/EtOAc 4:1) yields the title compound; MS: [M+1]⁺=478; tic (hexane/EtOAc 4:1) R_{f}=0.26.

### Example 20

### 2-f(6-Oxo-5-phenyl-1,6-dihydro-pyridin-3-ylmethyl)-amino]-N-(3-trifluoromethyl-phenyl)-benzamide

To a solution of 70 mg (0.15 mMol) of 2-[(6-methoxy-5-phenyl-pyridin-3-ylmethyl)-amino]-N-(3-trifluoromethyl-phenyl)-benzamide in 1.5 ml of CHCl₃ under N₂-atmosphere, 41 μl (0.30 mMol) of Me₃Sil are added. The mixture is stirred for 5 h at 60° C, cooled to RT and diluted with 5 ml of CHCl₃, 3 ml of sat. NaHCO₃ solution and 9 ml water. After stirring for 30 min, the aqueous layer is separated off and extracted twice with CH₂Cl₂. The organic phases are washed with water and brine, dried (Na₂SO₄) and concentrated. Column chromatography (SiO₂; CH₂Cl₂/acetone 2:1) yields the title compound; MS: [M+1]⁺=464.

### Example 21

### 2-[(5-Allyl-6-methoxy-pyridin-3-ylmethyl)-amino]-N-(3-trifluoromethyl-phenyl)-benzamide

A solution of 480 mg (1.00 mMol) of 2-[(5-bromo-6-methoxy-pyridin-3-ylmethyl)-amino]-N-(3-trifluoromethyl-phenyl)-benzamide (Expl. 17), 231 mg (0.20 mMol) of Pd(PPh₃)₄ and 1.95 g (5 mMol) of allyl-triphenyl-stannane (Fluka, Buchs/Schweiz) in 5 ml of degassed DMF are stirred for 20 h at 100° C. under a N₂-atmosphere. The resulting black suspension is diluted with water and EtOAc, the aqueous layer is separated off and extracted twice with EtOAc. The organic phases are washed with water and brine, dried (Na₂SO₄) and concentrated. Column chromatography (SiO₂; hexane/EtOAc 4:1) yields the title compound; MS: [M+1]⁺=442; tic (hexane/EtOAc 4:1) R_{f}=0.28.

### Example 22

### 2-[(5-ⁿPropyl-6-methoxy-pyridin-3-ylmethyl)-amino]-N-(3-trifluorometbyl-phenyl)-benzamide

A solution of 106 mg (0.24 mMol) of 2-[(5-allyl-6-metnoxy-pyridin-3-ylmethyl)-amino]-N-(3-trifluoromethyl-phenyl)-benzamide in 5 ml of methanol is hydrogenated in presence of Raney-Nickel. Filtration, concentration of the filtrate and column chromatography (SiO₂; hexane/CH₂Cl₂/Et₂O 250:250:1 →4 CH₂Cl₂/Et₂O 200:1) yields the title compound; MS: [M+1]⁺=444; tic (CH₂Cl₂/Et₂O 200:1) R_{f}=0.28.

### Example 23

### 2-[(5-Allyl-6-oxo-1.6-dihydro-pyridin-3-vimethyl)-amino]-N-(3-tnifluoromethyl-phenyl)-benzamide

Demethylation of 2-[(5-allyl-6-methoxy-pyridin-3-ylmethyl)-amino]-N-(3-trifluoromethyl-phenyl)-benzamide analoguesly to the procedure described in Expl. 20 yields the title compound; m.p. 155-158° C.; MS: [M+1]⁺=428; tic (EtOAc/hexane 2:1) R_{f}=0.13.

### Example 24

### 2-[(5-ⁿpropyl-6-oxo-1.6-dihydro-pyridin-ylmethyl)-amino]-N-(3-trifluoromethyl-phenyl)-benzamide

Demethylation of 2-[(5-ⁿ propyl-6-methoxy-pyddin-3-ylmethyl)-amino]-N-(3-trifluoromethyl-phenyl)-benzamide analoguesly to the procedure described in Expl. 20 yields the title compound; m.p. 151-153° C.; MS: [M+1]⁺=430.

### Example 25

### 2-[(5-Ethylamino-6-methoxy-pyridin-3-ylmethyl)-amino]-N-(3-trifluoromethyl-phenyl)-benzamide

A mixture of 500 mg (1.04 mMol) 2-[(5-bromo-6methoxy-pyridin-ylmethyl)-amino]-N-(3-trifluoromethyl-phenyl)-benzamide (Expl. 17), 62 mg R(+)-BINAP MR(+)-2,2′-bis-(diphenyl-phosphino)-1,1′-binaphthalin); 0.10 mMol], 27 mg Pd₂(dba)₃CHCl₃ [tris(dibenzylidene-acetone)dipalladium (0) chloroform complex; 0.026 mMol] and 200 mg (2.08 mnMol) of sodium-tert-butylate is prepared in 10 ml degassed DMF in a sealed tube under a N₂-atmoshere. Then 1.6 ml (3.1 mMol) of a 2 N solution of ethylamine in THF are added. After 70 h stirring at 70° C., the reaction mixture is diluted with EtOAc and sat. NaHCO₃ solution.

The separated aqueous layer is extracted twice with EtOAc, the organic phases washed with sat. NaHCO₃ solution, brine, dried (Na₂SO₄) and concentrated in vacuuo. Column chromatography (SiO₂; CH₂Cl₂/acetone 97:3) yields the title compound; MS: [M+1]⁺=445; tic (CH₂Cl₂/acetone 97:3) R_{f}=0.29; analysis for C,H,N,F (deviation ≦0.4%).

### Example 26

### 2-[(5-Ethylamino-6-oxo-1.6-dihydro-pyridin-3-ylmethyl)-amino]-N-(3-trifluoromethyl-phenyl)-benzamide

Demethylation of 2-[(5-ethylamino-6-methoxy-pyridin-3-ylmethyl)-amino]-N-(3-trifluoromethyl-phenyl)-benzamide analoguesly to the procedure described in Expl. 20 yields the title compound; MS: [M+1]⁺=431; analysis for C,H,N,F (deviation ≦0.4%).

### Example 27:

The following derivatives are obtained via above described procedures:

| | |
|---|---|
| | A |
| 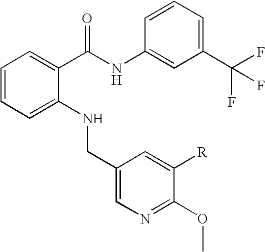 | |
| | B |
| 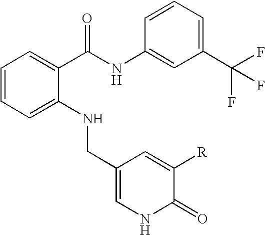 | |

| Ex. | 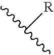 | Struct. type | Analog Expl. | MS: [M + 1]⁺ | m.p. [° C.] | Analysis¹ |
|---|---|---|---|---|---|---|
| 27AA 27AB | 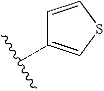 | A B | 19 20 | 484 470 | 151-152 | C, H, N, S, F |
| 27BA 27BB | 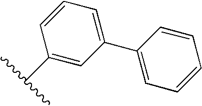 | A B | 19 20 | 554 540 | 141-142 | C, H, N, F |
| 27CA 27CB | 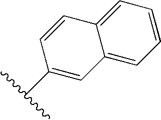 | A B | 19 20 | 528 514 | 158-162 | C, H, N, F |
| 27DA 27DB | 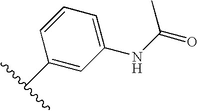 | A B | 19 20 | 535 521 | | |
| 27EA 27EB | 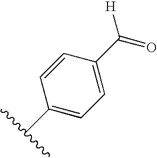 | A B | 19 20 | 506 492 | 136-137 | C, H, N, F |
| 27FA 27FB | 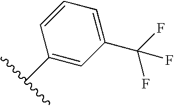 | A B | 19 20 | 546 532 | | C, H, N, F C, H, N, F |
| 27GA 27GB | 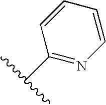 | A B | 21 20 | 479 465 | 150-151 215-216 | C, H, N, F |
| 27HA 27HB | 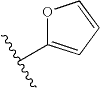 | A B | 21 20 | 468 454 | 143-144 | C, H, N, F |
| 27IA 27IB | 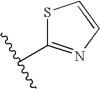 | A B | 21 20 | 485 471 | 152-153 200-201 | C, H, N, S, F |
| 27JA 27JB | 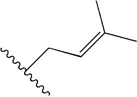 | A B | 21 | 470 | | |
| 27KA 27KB | 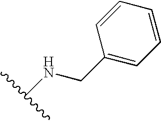 | A B | 25 20 | 507 493 | 182-186 | C, H, N, F C, H, N, F |
| 27LA 27LB | 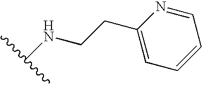 | A B | 25 20 | 522 508 | | |
| 27MA 27MB | 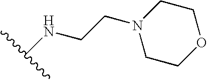 | A B | 25 20 | 530 516 | 173-176 | C, H, N, F C, H, N, F |
| 27NA 27NB | 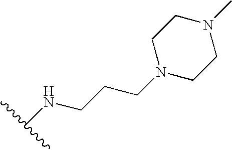 | A B | 25 20 | 557 543 | | |
| 27OA 27OB | 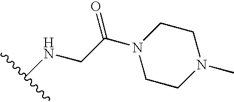 | A B | 25 20 | 557 543 | | C, H, N, F |

¹differences between calculated and experimental values ≦0.4%

### Example 28:

### 2-({6-Methoxy-5-[2-(4-methyl-Diperazin-1-yl)-ethylamino]-pyridin-3-ylmethyl}-amino)-N-(3-trifluoromethyl-phenyl)-benzamide

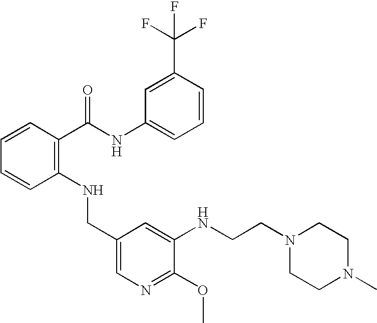

A solution of 500 mg (0.90 mMol) of 2-({6-methoxy-5-(2-(4-methyl-piperazin-1-yl)-2-oxo-ethylamino]-pyridin-3-ylmethyl}-amino)-N-(3-trifluoromethyl-phenyl)-benzamide (Expl. 27OA) in 20 ml of dichloromethane under an atmosphere of N₂ is cooled to −78° C. Then 4.2 ml of a 1 M solution of diisobutylaluminiumhydride in CH₂Cl₂ is added. After 1 h the mixture is slowly warmed up to −20° C. during 2.5 h. Then 15 ml of EtOAc are added, the mixture warmed up to RT and diluted with water and EtOAc. The, aqueous layer is separated off and extracted twice with EtOAc, the organic phases washed twice with water and brine, dried (Na₂SO₄) and concentrated in vacuuo. Column chromatography (SiO₂; dissolved in ethanol/acetone 1:1 and eluted with CH₂Cl₂/MeOH 9:1) yields crude product. Final distribution between EtOAc and water gives the title compound; MS: [M+1]⁺=543; tic (CH₂Cl₂/MeOH 9:1) R_{f}=0.10.

### Example 29

### 2-({5-[2-(4-Methyl-Diperazin-1-yl)-ethylamino]-6-oxo-1,6-dihydro-pyridin-3-ylmethyl}-amino)-N-(3-trifluornmethyl-phenyl)-benzamide

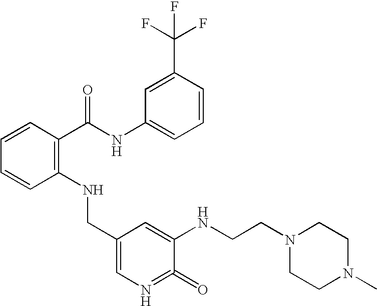

86 mg (0.16 mMol) of 2-({6-methoxy-5-[2-(4-methyl-piperazin-1-yl)-ethylamino]-pyridin-3-ylmethyl}-amino)-N-(3-trifluoromethyl-phenyl)-benzamide are converted to the title compound by cleavage with 75 μl of Me₃Sil as described in Expl. 20; MS: [M+1 ]⁺=529.

### Example 30

### 2-{[1,6-Dihydro-6-oxo-3-pyridinyl)methyl]amino}-N-(4-methyl-3-trifluoromethyl-phenyl)benzamide

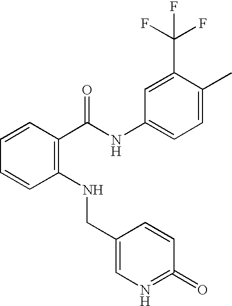

Analogously to Expl. 16, 500 mg (1.25 mMol) rac. 3-(4-methyl-3-trifluoromethyl-phenyl)-2-(6-oxo-1,6-dihydro-pyridin-3-yl)-2,3-dihydro-1H-quinazolin-4-one is reductively ring-opened with 0.60 ml (3.8 mMol) of triethylsilane and 0.60 ml (7.8 mMol) of trifluoroacetic acid in 2.3 ml of 1,2-dichloroethane. Crystallization from boiling acetonitrile finally gives the title compound; m.p. 232° C.; MS: [M+1]⁺=402.

### Step 30.1:

### N-(4-Methyl-3-trifluoromethyl-phenyl)-2-nitro-benzamide

The title compound is prepared analogously to step 1.1 by utilizing (4-methyl-3-trifluoromethyl)benzenamine and crystallized by partial concentration of a solution in EtOAc; m.p. 172-173° C.; MS: [M−1]=323.

### Step 30.2

### 2-Amino-N-(4-methyl-3-trifluoromethyl-phenyl)benzamide

Hydrogenation of 6.848 g (21.1 mMol) N-(4-methyl-3-trifluoromethyl-phenyl)-2-nitro-benzamide in 220 ml methanol in the presence of 0.7 g of Raney-Nickel, filtration and concentration of the filtrate gives the title compound; m.p. 138-140° C.; MS: [M+1]⁺=295.

### Step 30.3

### rac. 3-(4-Methyl-3-trifluoromethyl-phenyl)-2-(6-oxo-1,6-dihydro-pyridin-3-yl)-2,3-dihydro-1 H-quinazolin-4-one

5.98 g (20.3 mMol) of 2-amino-N(4-methyl-3-trifluoromethyl-phenyl)benzamide, 2.71 g (19.9 mMol) of 6-oxo-1,6-dihydro-pyridine-3-carbaldehyde (Step 16.1) and 14 mg of camphor-10-sulfonic acid in 70 ml of toluene are condensed analogously to Step 16.2. The crude product is dissolved in 0.6 l of boiling ethanol. Partial concentration leads to crystallization, yielding the title compound; m.p. 250-251° C.; MS: [M+1]⁺=400.

### Example 31

### 2-{[(1,6-Dihydro-6-oxo-3-pyridinyl)methyl]amino}-N-[3-(4-ethyl-piperazin-1-ylmethyl)-5-trifluoromethyl-phenyl]benzamide

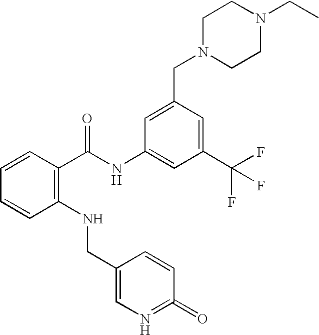

Analogously to Expl. 16, 189.6 mg (0.37 mMol) rac. 3-[3-(4-ethyl-piperazin-1-ylmethyl)-5-trifluoromethyl-phenyl]-2-(6-oxo-1,6-dihydro-pyridin-3-yl)-2,3-dihydro-1H-quinazolin-4-one is reductively ring-opened with 0.106 ml (0.68 mMol) of triethylsilane and 0.26 ml. (3.3 mmol) of trifluoroacetic acid in 3 ml of 1,2-dichloroethane, yielding the title compound; MS: [M+1]⁺=514.

### Step 31.1:

### (3-Nitro-5-trifluoromethyl-phenyl)-(4-ethyl-piperazin-1-yl)-methanone

In an ice bath under N₂-atmosphere, 11.8 g (50 mMol) of 3-nitro-5-trifluoromethyl-benzoic acid (Lancaster), 150 ml CH₂Cl₂, a few drops of DMF and 7.0 ml (81 mMol) of oxalylchloride are mixed and then stirred for 3 h at rt. The resulting solution is concentrated in vacuo. The residue is dissolved in 170 ml CH₂Cl₂ and added dropwise to an ice cooled solution of 12.7 ml (0.10 mol) N-ethyl-piperazine in 100 ml CH₂Cl₂. After stirring for 1 h at rt, the mixture is washed with a diluted solution of Na₂CO₃, 2 portions of water and finally brine. The aqueous layers are re-extracted twice with EtOAc, the combined organic phases dried (Na₂SO₄) and concentrated giving the title compound as an oil: MS: [M+1]⁺=332.

### Step 31.2:

### (3-Amino-5-trifluoromethyl-phenyl)-(4-ethyl-piperazin-1-yl)-methanone

Hydrogenation of 16.5 g (50 mMol) of (3-nitro-5-trifluoromethyl-phenyl)-(4-ethyl-piperazin-1-yl)-methanone in 300 ml ethanol-in the presence of 3 g of Raney-Nickel, filtration through celite, concentration of the filtrate and crystallization from hexane gives the title compound: m.p.: 104° C.; MS: [M+1]⁺=302.

### Step 31.3

### [3-(4-Ethyl-piperazin-1-ylmethyl)-5-trifluoromethyl]benzenamine

To a solution of 14.6 g, (48.5 mMol) (3-amino-5-trifluoromethyl-phenyl)-(4-ethyl-piperazin-1-yl)-methanone in 120 ml THF under N₂-atmosphere, 145.5 ml of a 1 M solution of BH₃-THF is added dropwise (exothermic). The mixture is stirred for 14 h at rt and then 5 h at 65° C. After cooling to rt, 250 ml of HCl conc./H₂O 1:1 is added and the mixture stirred for 15 h. The suspension is filtered, the filtrate extracted 3 times with EtOAc. The organic phases are washed twice with 1 N HCl and then discarded. The combined acidic phases are made basic by addition of saturated Na₂CO₃ solution and extracted 3 times with EtOAc. The organic layers are washed twice with water and brine, dried (Na₂SO₄) and concentrated. Column chromatography (SiO₂; EtOAc/EtOH/NH₃ 95:5:1→90:10:1) followed by extraction between 3 portions of EtOAc, 3 portions of water and finally brine, drying (Na₂SO₄) and concentration yields the title compound as an oil: MS: [M+1]⁺=288.

### Step 31.4

### N-[3-(4-Ethyl-piperazin-1-ylmethyl)-5-trifluoromethyl-phenyl]-2-nitro-benzamide

The title compound is prepared analogously to step 1.1 by utilizing [3-(4-ethyl-piperazin-1-ylmethyl)-5-trifluoromethyl]benzenamine; MS: [M+1]⁺=437.

### Step 31.5

### 2-Amino-N[3-(4-ethyl-piperazin-1-ylmethyl)-5-trifluoromethyl-phenyl]benzamide

Hydrogenation of 1.172 g (2.69 mMol) N-[3-(4-ethyl-piperazin-1-ylmethyl)-5-trifluoromethyl-phenyl]-2-nitro-benzamide in 35 ml methanol/THF 1:1 in the presence of 175 mg of Raney-Nickel, filtration and concentration of the filtrate gives the title compound; MS: [M+1]⁺=407.

### Step 31.6

### rac. 3-[3-(4-Ethyl-piperazin-1-ylmethyl)-5-trifluoromethyl-phenyl]-2-(6-oxo-1,6-dihydro-pyridin-3-yl)-2,3-dihydro-1 H-quinazolin-4-one

Under N₂-atmosphere, 637 mg (1.57 mMol) of 2-amino-N-[3-(4-ethyl-piperazin-1-ylmethyl)-5-trifluoromethyl-phenyl]benzamide, 189 mg (1.54 mMol) of 6-oxo-1,6-dihydro-pyridine-3-carbaldehyde (Step 16.1) and 801 mg (3.45 mMol) of camphor-10-sulfonic acid in 7 ml of toluene and 3.5 ml of tbutanol are stirred in an oil bath of 90° C. for 200 min. The reaction mixture is diluted with a Na₂CO₃ solution and EtOAc, the aqueous layer separated off and extracted twice with EtOAc. The organic phases are washed with water and brine, dried (Na₂SO₄) and after addition of SiO₂ concentrated in vacuuo. The resulting powder is put on top of a chromatography column (SiO_{2;} CH₂Cl2/MeOH 97:3). Eluation with CH₂Cl₂/MeOH/NH₃aq 97:3:0.5 →90:10:0.5 gives the title compound; MS: [M+1]⁺=512.

### Example 32

### 2-{[(1.6-Dihydro-6-oxo-3-pridinyl)methyl]amino}-N-[3-(azetidin-1-ylmethyl)-5-trifluoromethyl-phenyl]benzamide

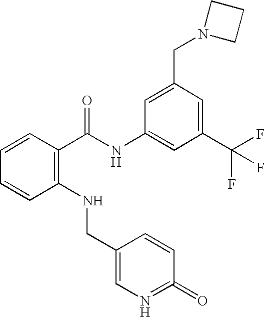

Under N₂-atmosphere, a mixture of 49 mg (0.10 mMol) of 2-[(6-methoxy-3-pyridinyl)methyl]amino-N-[3-(azetidin-1-ylmethyl)-5-trifluoromethyl-phenyl]benzamide and 47 mg (0.31 mMol) Nal in 1.0 ml CH₃CN and 1.8 IA (0.10 mMol) H₂O is heated to 60° C. 39 μl (0.31 mMol) Me₃SiCl are dropped in and stirring continued for 3 h. Then 0.23 ml MeOH are added at rt and the mixture is diluted with EtOAc and saturated NaHCO₃ solution. The aqueous layer is separated off and extracted twice with EtOAc. The organic phases are washed with a 10% solution of Na₂S₂O₃, water and brine, dried (Na₂SO₄) and concentrated. Reversed phase medium pressure liquid chromatography gives the title compound; MS: [M+1]⁺=457.

### Step 32.1

### (3-Nitro-5-trifluoromethyl-phenyl)-(azetidin-1-yl)-methanone

In an ice bath under N₂-atmosphere, 9.77 g (41.6 mMol) of 3-nitro-5-trifluoromethyl-benzoic acid (Lancaster), 150 ml CH₂Cl₂, a few drops of DMF and 5.8 ml (67 mMol) of oxalylchloride are mixed and then stirred for 17 h at rt. The resulting solution is concentrated in vacuo. The residue is dissolved in 50 ml CH₂Cl₂ and added dropwise to an ice cooled solution of 5.9 ml (87 mMol) azetidine in 50 ml CH₂Cl₂. After stirring for 15 min, the mixture is washed with 1 N HCI, a diluted solution of Na₂CO₃, water and brine. The aqueous layers are re-extracted twice with EtOAc, the combined organic phases dried (Na₂SO₄) and concentrated. Crystallization from hexane gives the title compound; m.p.: 91° C.; MS: [M+1]⁺=275.

### Step 32.2

### (3-Amino-5-trfluoromethyl-phenyl)-(azetidin-1-yt)-methanone

Hydrogenation of 10.39 g (37.9 mMol) of (3-nitro-5-trifluoromethyl-phenyl)-(azetidin-1-yl)-methanone in 200 ml ethanol in the presence of 2 g of Raney-Nickel, filtration through celite, partial concentration of the filtrate and dilution with hexane gives the crystalline title compound; m.p.: 154° C.; MS: [M+1]⁺=245.

### Step 32.3

### [3-(Azetidin-1-ylmethyl)-5-trifluoromethyl]benzenamine

To 8.62 g (35.3 mMol) (3-amino-5-trifluoromethyl-phenyl)-(azetidin-1-yl)-methanone in 75 ml THF under N₂-atmosphere cooled in an ice-bath, 10.6 ml (95%; 106 mMol) of BH₃/Me₂S in 15 ml THF are added dropwise. The resulting solution is stirred for 2 days at rt and then 4 h at 65° C. After cooling to rt, 50 ml of HCI conc./H₂O 1:1 is added and the mixture stirred for 15 h at rt and 7 h at 65° C. The mixture is poured off into EtOAc and a 10% solution of Na₂CO₃, the aqueous phase separated off and extracted twice with EtOAc. The organic layers are washed twice with water and brine, dried (Na₂SO₄) and concentrated. Column chromatography (SiO₂; EtOAc/EtOH 95:5→EtOAc/EtOH/Et₃N 95:5:14) yields the title compound; m.p. 60-61° C.; MS: [M+1 ]⁺=231.

### Step 32.4

### N-13-(Azetidin-1-ylmethyl)-5-trifluoromethyl-phenyl]-2-nitro-benzamide

A solution of 770 mg (3.34 mMol) of 3-(azetidin-1-ylmethyl)-5-trifluoromethyl]benzenamine in 15 ml CH₂Cl₂ and 3 ml pyridine is cooled to -70° C. under a N₂-atmosphere. Then a solution of 651 mg (3.5 mMol) of 2-nitrobenzoyl chloride (Fluka, Buchs, Switzerland) in 15 ml CH₂Cl₂ is added dropwise. After stirring for 60 min, the resulting mixture is diluted with EtOAc and water, the aqueous layer separated off and extracted twice with EtOAc. The organic phases are washed with water and brine, dried (Na₂SO₄) and concentrated in vacuuo. Column chromatography (SiO₂; CH₂Cl₂/MeOH 99:1 <CH₂Cl₂/MeOH/NH₃aq 99:1:1) gives the title compound; MS: [M+1 ]⁺=380.

### Step 32.5

### 2-Amino-AP[3-(azefidin-1-ylmethyi)-5-trifluoromethyl-phenyl]benzamide

Hydrogenation of 490 mg (1.29 mMol) N-[3-(azetidin-1-ylmethyl)-5-trifluoromethyl-phenyl]-2-nitro-benzamide in 15 ml methanol/THF 1:1 in the presence of 75 mg of Raney-Nickel, filtration and concentration of the filtrate gives the title compound; MS: [M+1 ]⁺=350.

### Step 32.6

### 2-[(6-Methoxy-3-pyridinyl)methyl]amino-N-[3-(azetidin-1-ylmethyl)-5-trifluoromethyl-phenyl]benzamide

Prepared analogously to Example 1 from 6-methoxy-3-pyridinecarbaldehyde and 2-amino-N-[3-(azefidin-1-ylmethyl)-5-trifluoromethyl-phenyl]benzamide; MS: [M+1]⁺=471.

### Example 33

### 2-f(6-Methoxy-3-pyridinyl)methyl]amino-N-[4-(4-methyl-Diperazin-l-ylmethyl)-3-trifluoromethyl-phenyl[benzamide

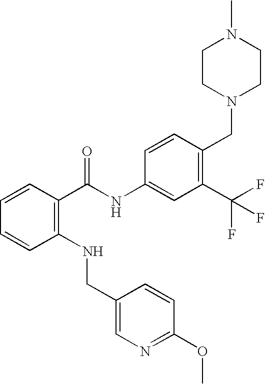

To a solution of 545.3 mg (1.39 mMol) of 2-amino-N[4-(4methyl-piperazin-1-ylmethyl)-3-trifluoromethyl-phenyl]benzamide in 14.2 ml of MeOH/CH₃COOH 99:1 under N₂-atmosphere, 286 mg (2.08 mMol) 6-methoxy-3-pyridinecarboxaldehyde (Fluka, Buchs, Switzerland) are given. After 15 min, 304 mg (4.8 mMol) of NaBH₃CN are added and the mixture is stirred for 20 h at r.t. The concentrated reaction mixture is dissolved in CH₂Cl₂ and a saturated NaHCO₃ solution, the aqueous layer separated off and extracted twice with CH₂Cl₂. The organic phases are washed with H₂O and brine, dried (Na₂SO₄) and concentrated. Medium pressure liquide chromatography (SiO₂; CH₂Cl₂/MeOH+1% NH₃^{aq} 95:5→4:1) yields the title compound; MS: [M+1]⁺=514.

### Step 33.1

### N-(4-Methyl-3-trifluoromethyl-phenyl)-2,2,2-trifluoro-acetamide

To an ice-cooled solution of 20.2 g (112 mMol) of 5-amino-2-methylbenzotrifluoride and 89.8 ml (1.12 Mol) pyridine in 285 ml of CH₂Cl₂ under N₂-atmosphere, 17.1 ml (123 mMol) of trifluoroacetic acid anhydride are added dropwise. After 20 min, the mixture is diluted with 0.7 l CH₂Cl₂ and washed with a 10% solution of citric acid in water, twice with water and finally brine. The aqueous layers are extracted twice with CH₂Cl₂, the organic phases dried (Na₂SO₄) and concentrated partially. Crystallization by addition of hexane yields the title compound; m.p.: 72-73° C.

### Step 33.2

### 2 N-(4-Bromomethyl-3-trifluoromethyl-phenyl)-2,2,2-trifluoro-acetamide

To a solution of 60.9 g (224.6 mMol) of N-(4-methyl-3-trifluoromethyl-phenyl)-2,2,2-trifluoro-acetamide in 830 ml nbutyl acetate under N₂-atmosphere, 44 g (247 mMol) N-bromosuccinimide and 830 mg (5 mMol) azo-iso-butyronitrile are added. The suspension is heated up to 60° C. and then illuminated for 30 min by a Phillips low-voltage lamp (500 W; 10500 lm), whereby the temperature rises to 70-75° C. and a clear brown solution is formed. There is still remaining educt, therefore another 22 g N-bromosuccinimide are added in 3 portions. After totally 6 h, the resulting suspension is filtered off and the filtrate concentrated. The residue is distributed between 2 |CH₂Cl₂ and 1 |H₂O and the aqueous layer extracted with 1 |CH₂Cl₂. The organic phases are washed 4 times with 1 |H₂O, 0.5 |brine, dried (Na₂SO₄) and concentrated. Column chromatography (SiO₂; hexane/CH₂Cl₂ 2:1-1:1) and crystallization from CH₂Cl₂/hexane yields the title compound; m.p.: 119-120°′.

### Step 33.3

### 2,2,2-Trifluoro-N-[4-(4-methyl-piperazin-1-ylmethyl)-3-trifluoromethyl-phenyl]-acetamide

To an ice-cooled solution of 1.9 ml (17.1 mMol) N-methylpiperazine in 50 ml acetonitril under N₂-atmosphere, a solution of 2.00 g (5.71 mMol) N-(4-bromomethyl-3-trifluoromethyl-phenyl)-2,2,2-trifluoro-acetamide in 50 ml acetonitril is added dropwise during 30 min. After additional 20 min, the reaction mixture is concentrated in vacuo. The resulting oil is diluted with EtOAc and saturated NaHCO₃-solution/H₂O 1:1. The aqueous layer is separated off and extracted twice with EtOAc. The organic layers are washed with saturated NaHCO₃-solution/H₂O 1:1, water and brine, dried (Na₂SO₄), concentrated and directly used in Step 33.4: MS: [M+1]⁺=370.

### Step 33.4

### [[4-(4-Methyl-piperazin-1-ylmethyl)-3-trifluoromethyl]benzenamine

To a solution of 1.102 g (2.98 mmol) of 2,2,2-trifluoro-N-[4-(4-methyl-piperazin-1-ylmethyl)-3-trifluoromethyl-phenyl]-acetamide in 26 ml of boiling methanol, 14 ml of a 1 M solution of K₂CO₃ in water are added dropwise. After 1 h stirring, the reaction mixture is cooled to rt and diluted with EtOAc and water. The aqueous layer is separated off and extracted twice with EtOAc. The organic phases are washed with water and brine, dried (Na₂SO₄) and concentrated to yield the title compound, which was directly used in Step 33.5: MS: [M+1 ]⁺=274.

### Step 33.5

### N-[4(4-Methyl-piperazin-1-ylmethyl)-3-trifluoromethyl-phenyl]-2-nitro-benzamide

A solution of 740 mg (2.71 mMol) of 4-(4-Methyl-piperazin-1-ylmethyl)-3-trifluoromethyl]benzenamine in 12.5 ml CH₂Cl₂ and 2.5 ml pyridine is cooled in an ice-bath under N₂-atmosphere. Then a solution of 375 pl (2.85 mMol) of 2-nitrobenzoyl chloride (Fluka, Buchs, Switzerland) in 12.5 ml CH₂Cl₂ is added dropwise. After 30 min, the resulting mixture is diluted with EtOAc and 0.5 N HCI, the aqueous layer separated off and extracted with EtOAc. The organic phases are washed 3 times with 0.5 N HCl and then discarded. The combined aqueous phases are turned basic by the addition of saturated Na₂CO₃ solution and extracted with 3 portions of EtOAc. The organic phases are washed with brine, dried (Na₂SO₄) and concentrated in vacuuo. Column chromatography (SiO_{2;} EtOAc →EtOAc/EtOH(+1% Et³N) 97:3) gives the title compound; MS: [M+1]⁺=423.

### Step 33.6

### 2-Amino-N-[4-(4-methyl-piperazin-1-ylmethyl)-3-trifluoromethyl-phenyl]benzamide

Hydrogenation of 378 mg (0.89 mMol) N-[4-(4-methyl-piperazin-1-ylmethyl)-3-trifluoromethyl-phenyl-2-nitro-benzamide in 17 ml methanol in the presence of Raney-Nickel, filtration and concentration of the filtrate gives the title compound; MS: [M+1]⁺=393.

### Example 34

### 2-{[(1,6-Dihydro-6-oxo-3-pyridinyl)methyl]amino}-N-[4-(4-methyl-piperazin-l-ylmethyl)-3trifluoromethyl-phenyl]benzamide

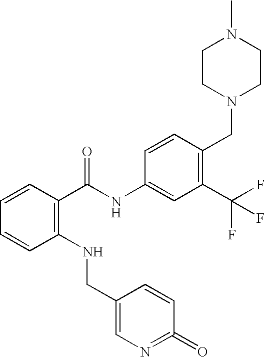

Analogously to Example 32, 2-[(6-methoxy-3-pyridinyl)methyl]amino-N-[4-(4-methyl-piperazin-1-ylmethyl)-3-trifluoromethyl-phenyl]benzamide is cleaved to the title compound by Me₃SiCl, Nal and H₂O; MS: [M+1]⁺=500.

### Example 35

The following derivatives can be obtained via above described procedures:

| | |
|---|---|
| | A |
| 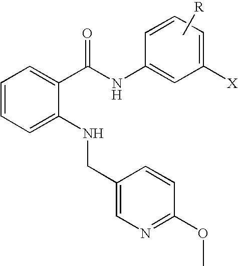 | |
| | B |
| 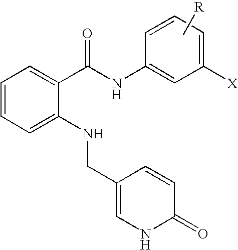 | |

| Ex. | 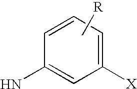 | Struct. type | MS: [M + 1]⁺ |
|---|---|---|---|
| 35AA 35AB | 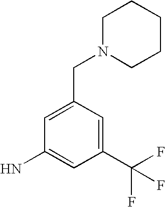 | A B | 499 485 |
| 35BA 35BB | 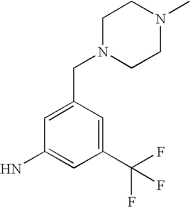 | A B | |
| 35CA 35CB | 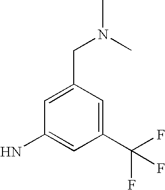 | A B | |
| 35DA 35DB | 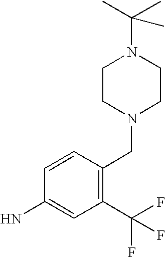 | A B | |
| 35EA 35EB | 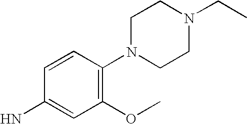 | A B | |
| 35FA 35FB | 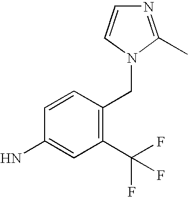 | A B | |

### Example 36

### 2-{[(1.6-Dihydro-6-oxo-3-pyridinyl)methyl]amino}-N-4-[[2-(dimethylamino)ethyl]methylamino]-3-trifluoromethyl-phenyl]benzamide

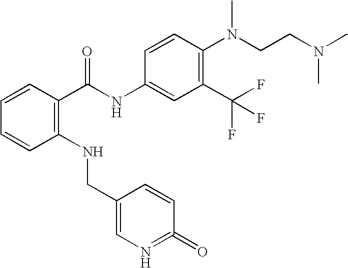

A solution of 6-oxo-1,6-dihydropyridine-3-carboxaldehyde (123 mg, 1.0 mmol), 2-amino-N-4-[[2-(dimethylamino)ethyl]methylamino]-3-trifluoromethyl-phenyl]benzamide (460 mg, 1.0 mmol) and camphor-10-sulfonic acid (2 mg) in dry toluene (35 mL) is heated under reflux for 24 h, with the water formed during the reaction being removed by employing a Soxlet apparatus charged with 4A molecular sieves. The solvent is then evaporated off under reduced pressure and the residue is dissolved in methanol (5 mL). The resulting solution is added to a mixture of sodium cyanoborohydride (400 mg of 90%, 5.75 mmol) and acetic acid (0.5 mL) in methanol (45 mL) and stirred at 20° C. for 1 h. The methanol is evaporated off under reduced pressure to afford a residue which is dissolved in ethyl acetate (100 mL). The mixture is washed with a saturated aqueous solution of sodium hydrogen carbonate (2×50 mL), dried (Na₂SO₄), filtered and the solvent is evaporated off under reduced pressure to yield the crude product which is purified by column chromatography on silica gel, eluent 12% methanol in dichloromethane and recrystallised from methanol to give the title 6ompound as a pale-yellow crystalline solid, m.p. 222-225° C.

### Step 36.1

### M N. N-Trimethyl-N-(4-nitro-2-trifluoromethylphenyl)-1,2-ethanediamine

A mixture of 2-bromo-5-nitrobenzotrifuoride (Lancaster Synthesis, GmbH; 5.4 g, 20 mmol) and N,N,N-trimethylethylenediamine (Fluka, Buchs, Switzerland; 2.25 g, 22 mmol) and N,N-diisopropylethylamine (13.9 mL) is heated at 80° C. for 18 hours in a steel pressure vessel. The mixture is then cooled, treated with a saturated aqueous solution of sodium hydrogen carbonate (50 mL) and extracted with ethyl acetate (2×100 mL). The combined extracts are dried (Na₂SO₄), filtered and the solvent is evaporated off under reduced pressure to yield the crude product which is used directly without further purification.

### Step 36.2

### Nr-[2-(Dimethylamino)ethyl]-N-methyl-3-trifluoromethyl-1,4-benzenediamine

The title compound is prepared by a method analogous to that described in Example 1.2 utilising N,N,N-trimethyl-N-(4-nitro-2-trifluoromethylphenyl)-1 ,2-ethanediamine in lieu of 2-nitro-N-(4-bromo-3-trifluoromethylphenyl]benzamide.

### Step 36.3

### 2-Nitro-N-4-[2-(dimethylamino)ethyl]methylamino]-3-trifluoromethyl-phenyl]benzamide

A stirred solution of N-[2-(dimethylamino)ethyl]-N-methyl-3-trifluoromethyl-1,4-benzenediamine (1.31 g, 5 mmol) in toluene. (80 mL) under an argon atmosphere at 0° C., is treated with trimethylaluminium (5 mL of a 2 M solution in toluene, 10 mmol) and stirred at 20° C. for 1 h. A solution of 2-nitrobenzoic acid, methyl ester (Fluka, Buchs, Switzerland; 0.91 g, 5 mmol) in dry toluene (40 mL) is then added and the mixture is stirred 110° C. for 4 h. The cooled mixture is then treated with hydrochloric acid (50 mL of 1 M), stirred for 15 min and then treated with aqueous sodium hydroxide (50 mL of 1 M). The mixture is then treated with water (100 mL) and extracted with ethyl acetate (3×100 mL). The combined extacts are washed with brine (2×50 mL), dried (MgSO₄) and the solvent is evaporated off under reduced pressure to give the crude product which is used directly without further purification.

### Step 36.4

### 2-Amino-N-4-[[2-(dimethylamino)ethyl]methylamino]-3-trifluoromethyl-phenyl]benzamide

The title compound is prepared by a method analogous to that described in Example 1.2 utilising 2-nitro-N-4-1[2-(dimethylamino)ethyl]methylamino]-3-trifluoromethyl-phenyl]benzamide in lieu of 2-nitro-N-(4-bromo-3-trifluoromethylphenyl)benzamide.

### Example 37

### 2-{[1 .6-Dihydro-6-oxo-3-pyridinyl)methyl]amino}-N5-(5-Methyl-1 H-imidazol-1-yl)-3-trifluoromethyl-phenyl]benzamide

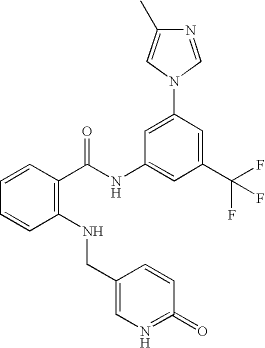

The title compound is prepared by a method analogous to that described in Example 36 utilising 2-amino-N-5-(5-methyl-1 H-imidazol-1-yl)-3-trifluoromethyl-phenyl]benzamide in lieu of 2-amino-N4-[[2-(dimethylamino)ethyl]methylamino]-3-trffluoromethyl-phenyl]benzamide; m.p. 192-195° C.

### Step 37.a:

### 3-(2-Methyl-lH-imidazol-1-yl)-5-(trifluoromethyl)-benzonitrile

A mixture of 3-fluoro-5-(trifluoromethyl)-benzonitrile (Lancaster Synthesis GmbH; 17 g, 89 mmol) and 2-methylimidazole (Fluka, Buchs, Switzerland; 22.2 g, 270 mmol) in N,N-dimethylacetamide (80 mL) is stirred at 145° C. for 19 h. The solvent is evaporated off under reduced pressure and the residue is dissolved in ethyl acetate (200 mL). The solution is washed with brine (200 mL), dried (Na₂SO₄) and the solvent is evaporated off under reduced pressure to give the crude product which is purified by recrystallisation from ether-hexane to afford the title compound as yellow crystalline solid, m.p. 132-134° C.

### Step 37.b

### 3-(2-Methyl-1 H-imidazol-1-yl)-5-(trifluoromethyl)-benzoic acid

A solution of 3-(2-methyl-1 H-imidazol-1-yl)-5-(trifluoromethyl)-benzonitrile (Example 91a; 16.7 g, 66 mmol) in dioxane (300 mL) is added to an aqueous solution of sodium hydroxide (275 mL of 1 M) and the mixture is heated at 95° C. for 18 h. The solvent is evaporated off under reduced pressure and the residue is neutralised with hydrochloric acid (1 M) and extracted with butanol (2×250 mL). The solvent is evaporated of under reduced pressure to give the title compound. ¹H-NMR (400 MHz, DMSO-d₆, Δ): 7.17 (s,1 H); 8.03 (s,1 H); 8.12 (s,1H); 8.35 (s, 1H); 8.41 (s, 1H); 8.53 (s, 1H); 13.90 (br., 1H).

### Step 37.c

### [3-(2-Methyl-1 H-imidazol-1-yl)-5-(trifluoromethyl)phenyl]-carbamic acid, 1,1-dimethylethyl ester

Triethylamine (5.23 mL, 37.5 mmol) is added to a stirred suspension of 3-(2-methyl-1 H-imidazol-1-yl)-5-(trifluoromethyl)-benzoic acid (6.8 g, 25 mmol) in t-butanol (200 mL). Diphenylphosphorylazide (7.6 g, 27.5 mmol) is added to the resulting solution and the mixture is heated 80° C. for 16 h. The solvent is evaporated off under reduced pressure and the residue is treated with water (100 mL) and extracted with ethyl acetate (2×100 mL). The combined extracts are washed with brine (100 mL), dried (Na₂SO₄) and the solvent is evaporated off under reduced pressure to give the crude product which is purified by column chromatography (silica gel, eluent 2% ethanol in ethyl acetate) and recrystallised from ether-hexane to afford the title compound as a colourless crystalline solid, m.p. 203-208° C.

### Step 37.d:

### 5-(2-Methyl-1 H-imidazol-1-yl)-3-(trifluoromethyl)-benzenamine

[3-(2-Methyl-1 H-imidazol-1-yl)-5-(trifluoromethyl)phenyl]-carbamic acid, 1,1-dimethylethyl ester (9.1 g, 26.7 mmol) is treated with a solution of hydrogen chloride in isopropanol (100 mL of 4 M) and heated at 55° C. for 15 min. The cooled mixture is treated with aqueous sodium hydroxide solution (100 mL of 4 M) and extracted with ethyl acetate (3×100 mL). The combined extacts are washed with brine (2×100 mL), dried (MgSO₄) and the solvent is evaporated off under reduced pressure to give the crude product which is purified by recrystallisation from ethyl acetate-hexane to afford the title compound as colourless crystalline solid, m.p. 130-133° C.

### Step 37.e:

### 2-Nitro-N-5-(5-Methyl-1 H-imidazol-1-yl)-3-trifluoromethyl-phenyl]benzamide

The title compound is prepared by a method analogous to that described in Example 36.3 utilising 5-(2-methyl-1 H-imidazol-1-yl)-3-(trifluoromethyl)-benzenamine in lieu of N-[2-(dimethylamino)ethyl]-N-methyl-3trifluoromethyl-1,4-benzenediamine; m.p. 200-201° C.

### Step 37.f:

### 2-Amino-N-5-(5-Methyl-1 H-imidazol-1-yl)-3-trifluoromethyl-phenyl]benzamide

The title compound is prepared by a method analogous to that described in Example 1.2 utilising 2-nitro-N-(5-Methyl-1 H-imidazol-1-yl)-3-trifluoromethyl-phenyl]benzamide in lieu of 2-nitro-N-(4-bromo-3-trifluoromethylphenyl)benzamide; m.p. 268-270° C.

### Example 38:

### Soft capsules

5000 soft gelatin capsules, each comprising as active ingredient 0.05 g of one of the compounds of formula I mentioned in the preceding Examples, are prepared as follows:

| | | |
|---|---|---|
| Active ingredient | 250 | g |
| Lauroglycol | 2 | litres |

Preparation process: The pulverized active ingredient is suspended in Lauroglykol® (propylene glycol laurate, GattefosséS.A., Saint Priest, France) and ground in a wet pulverizer to produce a particle size of about 1 to 3 μm. 0.419 g portions of the mixture are then introduced into soft gelatin capsules using a capsule-filling machine.

1-16. (canceled)

17. An anthranilic acid amide of formula I, 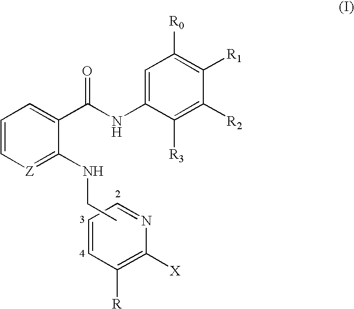
wherein
R and R₀ represent H, halogen, alkynyl, alkenyl, alkyl, which in each case is unsubstituted or substituted by halogen; unsubstituted or substituted mono- or bicyclic aryl; unsubstituted or substituted mono- or bicyclic heteroaryl having 1 to 3 heteroatoms selected from O, N or S; unsubstituted or substituted heterocyclyl having at least one N atom; mono- or dialkyl amino, wherein the alkyl radical is unsubstituted or substituted by unsubstituted or substituted aryl, unsubstituted or substituted mono- or bicyclic heteroaryl having 1 to 3 heteroatoms selected from O, N or S or substituted by unsubstituted or substituted heterocyclyl having at least one N atom; unsubstituted or substituted heterocyclyl carbonyl alkyl amino, wherein the heterocyclyl radical comprises at least one N atom;
R₁ represents H, halogen, unsubstituted or substituted C₁₋₇alkyl, C₂₋₇alkenyl, C₂₋₇alkynyl, alkoxy or a radical —O-(CH₂)ₙ-CF₃, wherein n is 0, 1, 2 or 3,
R₂ is perfluoro alkyl,
R₃ represents H or halogen,
X represents hydroxy, alkoxy, alkyl thio, imino, alkyl imino, halogen, a radical of formula I′ 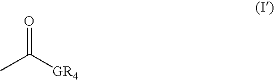
wherein G is CH₂ or NH and R₄ is hydrogen, alkyl or aryl, or a radical of formula I″ 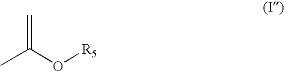
wherein R₅ is alkyl or aryl,
Z is N or CH, and
wherein the methylen group is attached to the pyridyl moiety at the carbon atom of the pyridyl moiety in 2-, 3-, 4- or 5-position,
under the proviso that R cannot represent H, if Z is nitrogen, X is hydroxy or methoxy and wherein the methylen group is attached to the pyridyl moiety at the carbon atom of the pyridyl moiety in 3-position, R₁ and R₃ cannot both represent H if Z is CH, R represents H, X is hydroxy, alkoxy or alkyl thio and wherein the methylen group is attached to the pyridyl moiety at the carbon atom of the pyridyl moiety in 3-position, and R₁ and R₃ cannot both represent H if Z is CH, R and R₀ both represent H, R₂ represents trifluoromethyl, X is bromo or hydroxy and wherein the methylen group is attached to the pyridyl moiety at the carbon atom of the pyridyl moiety in 4-position,
or an N-oxide or a tautomer thereof,
or a salt of such anthranilic acid amide, its N-oxide or its tautomer.

18. An anthranilic acid amide of formula I according to claim 17, wherein
R represents H, halogen, alkynyl, alkenyl, alkyl, which in each case is unsubstituted or substituted by halogen; unsubstituted or substituted mono- or bicyclic aryl; unsubstituted or substituted mono- or bicyclic heteroaryl having 1 to 3 heteroatoms selected from O, N or S; unsubstituted or substituted heterocyclyl having at least one N atom; mono- or dialkyl amino, wherein the alkyl radical is unsubstituted or substituted by unsubstituted or substituted aryl, unsubstituted or substituted mono- or bicyclic heteroaryl having 1 to 3 heteroatoms selected from O, N or S or substituted by unsubstituted or substituted heterocyclyl having at least one N atom; unsubstituted or substituted heterocyclyl carbonyl alkyl amino, wherein the heterocyclyl radical comprises at least one N atom;
R₀ represents H,
R₁ represents H, halogen, C₂₋₇alkyl, C₂₋₇alkenyl, C₂₋₇alkynyl, alkoxy or a radical —O-(CH₂)ₙ-CF₃, wherein n is 0, 1, 2 or 3,
R₂ is perfluoro alkyl,
R₃ represents H or halogen,
X represents hydroxy, alkoxy, alkyl thio, imino, alkyl imino, halogen, a radical of formula I′ 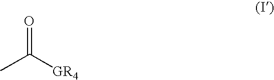
wherein G is CH₂ or NH and R₄ is hydrogen, alkyl or aryl, or a radical of formula I″ 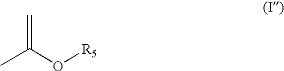
wherein R₅ is alkyl or aryl,
Z is N or CH, and
wherein the methylen group is attached to the pyridyl moiety at the carbon atom of the pyridyl moiety in 2-, 3-, 4- or 5-position,
under the proviso that R cannot represent H, if Z is nitrogen, X is hydroxy or methoxy and wherein the methylen group is attached to the pyridyl moiety at the carbon atom of the pyridyl moiety in 3-position, R. and R₃ cannot both represent H if Z is CH, R represents H, X is hydroxy, alkoxy or alkyl thio and wherein the methylen group is attached to the pyridyl moiety at the carbon atom of the pyridyl moiety in 3-position, R₁ and R₃ cannot both represent H if Z is CH, R represent H, R₂ represents trifluoromethyl, X is bromo or hydroxy and wherein the methylen group is attached to the pyridyl moiety at the carbon atom of the pyridyl moiety in 4-position,
or an N-oxide or a tautomer thereof,
or a salt of such anthranilic acid amide, its N-oxide or its tautomer.

19. An anthranilic acid amide of formula I according to claim 17 wherein
R represents H, halogen, alkenyl, alkyl, pyridyl alkyl amino, morpholinyl alkyl amino, alkyl piperazinyl alkyl amino, alkyl piperazinyl carbonyl alkyl amino, phenyl alkyl amino, alkyl amino, thienyl, pyridyl, furanyl, thiazolyl, naphthyl or phenyl which is unsubstituted or substituted by trifluoromethyl, phenyl, alkanoyl or alkanoyl amino,
R₁ represents H, halogen, C₂₋₇alkyl, C₂₋₇alkenyl, C₂₋₇alkynyl, alkoxy or a radical —O-(CH₂)ₙ -CF₃, wherein n is 0, 1, 2 or 3,
R₂ is perfluoro alkyl,
R₃ represents H or halogen,
X represents hydroxy, alkoxy, alkyl thio, imino, alkyl imino, halogen, a radical of formula I′ wherein G is CH₂ or NH and R₄ is hydrogen or alkyl, or a radical of formula I″ wherein R₅ is alkyl,
Z is N or CH, and
wherein the methylen group is attached to the pyridyl moiety at the carbon atom of the pyridyl moiety in 2-, 3-, 4- or 5-position,
under the proviso that R cannot represent H, if Z is nitrogen, X is hydroxy or methoxy and wherein the methylen group is attached to the pyridyl moiety at the carbon atom of the pyridyl moiety in 3-position, R₁ and R₃ cannot both represent H if Z is CH, R represents H, X is hydroxy, alkoxy or alkyl thio and wherein the methylen group is attached to the pyridyl moiety at the carbon atom of the pyridyl moiety in 3-position, R₁ and R₃ cannot both represent H if Z is CH, R and R₀ both represent H, R₂ represents trifluoromethyl, X is bromo or hydroxy and wherein the methylen group is attached to the pyridyl moiety at the carbon atom of the pyridyl moiety in 4-position,
or an N-oxide or a tautomer thereof,
or a salt of such anthranilic acid amide, its N-oxide or its tautomer.

20. An anthranilic acid amide of formula I according to claim 17, wherein
R represents H, halogen, lower alkenyl, lower alkyl, pyridyl lower alkyl amino, morpholinyl lower alkyl amino, lower alkyl piperazinyl lower alkyl amino, lower alkyl piperazinyl carbonyl lower alkyl amino, phenyl lower alkyl amino, lower alkyl amino, thienyl, pyridyl, furanyl, thiazolyl, naphthyl or phenyl which is unsubstituted or substituted by trifluoromethyl, phenyl, lower alkanoyl or lower alkanoyl amino,
R₁ represents H, halogen, C₂₋₇alkyl, C₂₋₇alkenyl, C₂₋₇alkynyl, lower alkoxy or a radical —O-(CH₂)ₙ-CF₃, wherein n is 0, 1, 2 or 3,
R₂ is trifluoromethyl,
R₃ represents H or halogen,
X represents hydroxy, lower alkoxy, lower alkyl thio, imino, lower alkyl imino, halogen, a radical of formula I′ wherein G is CH₂ or NH and R₄ is hydrogen or lower alkyl, or a radical of formula I″ wherein R₅ is lower alkyl,
Z is N or CH, and
wherein the methylen group is attached to the pyridyl moiety at the carbon atom of the pyridyl moiety in 2-, 3-, 4- or 5-position,
under the proviso that R cannot represent H, if Z is nitrogen, X is hydroxy or methoxy and wherein the methylen group is attached to the pyridyl moiety at the carbon atom of the pyridyl moiety in 3-position, R₁ and R₃ cannot both represent H if Z is CH, R represents H, X is hydroxy, lower alkoxy or lower alkyl thio and wherein the methylen group is attached to the pyridyl moiety at the carbon atom of the pyridyl moiety in 3-position, R₁ and R₃ cannot both represent H if Z is CH, R and Ro both represent H, X is bromo or hydroxy and wherein the methylen group is attached to the pyridyl moiety at the carbon atom of the pyridyl moiety in 4-position,
or an N-oxide or a tautomer thereof,
or a salt of such anthranilic acid amide, its N-oxide or its tautomer.

21. An anthranilic acid amide of formula I according to claim 17, wherein
R represents H, halogen, lower alkenyl, lower alkyl, pyridyl lower alkyl amino, morpholinyl lower alkyl amino, lower alkyl piperazinyl lower alkyl amino, lower alkyl piperazinyl carbonyl lower alkyl amino, phenyl lower alkyl amino, lower alkyl amino, thienyl, pyridyl, furanyl, thiazolyl, naphthyl or phenyl which is unsubstituted or substituted by trifluoromethyl, phenyl, lower alkanoyl or lower alkanoyl amino, R₁ represents H, halogen, C₂₋₇alkyl, C₂₋₇alkenyl, C₂₋₇alkynyl, lower alkoxy or a radical —O-(CH₂),-CF₃, wherein n is 0 or 1,
R₂ is trifluoromethyl,
R₃ represents H or halogen,
X represents hydroxy, lower alkoxy, halogen, a radical of formula I′ wherein R4 is hydrogen or lower alkyl, or a radical of formula I″ wherein R₅ is lower alkyl,
Z is N or CH, and
wherein the methylen group is attached to the pyridyl moiety at the carbon atom of the pyridyl moiety in 3- or 4-position,
under the proviso that R cannot represent H, if Z is nitrogen, X is hydroxy or methoxy and wherein the methylen group is attached to the pyridyl moiety at the carbon atom of the pyridyl moiety in 3-position, R₁ and R₃ cannot both represent H if Z is CH, R represents H, X is hydroxy, lower alkoxy or lower alkyl thio and wherein the methylen group is attached to the pyridyl moiety at the carbon atom of the pyridyl moiety in 3-position, R₁ and R₃ cannot both represent H if Z is CH, R and Ro both represent H, X is bromo or hydroxy and wherein the methylen group is attached to the pyridyl moiety at the carbon atom of the pyridyl moiety in 4-position,
or an N-oxide or a tautomer thereof,
or a salt of such anthranilic acid amide, its N-oxide or its tautomer.

22. An anthranilic acid amide of formula I according to claim 17, wherein R represents H, halogen, lower alkenyl, lower alkyl, pyridyl lower alkyl amino, morpholinyl lower alkyl amino, lower alkyl piperazinyl lower alkyl amino, lower alkyl piperazinyl carbonyl lower alkyl amino, phenyl lower alkyl amino, lower alkyl amino, thienyl, pyridyl, furanyl, thiazolyl, naphthyl or phenyl which is unsubstituted or substituted by trifluoromethyl, phenyl, lower alkanoyl or lower alkanoyl amino,
R₁ represents H, halogen, C₂₋₇alkyl, or C₂₋₇alkynyl,
R₂ is trifluoromethyl,
R₃ represents H or halogen,
X represents hydroxy, lower alkoxy, halogen, a radical of formula I′ wherein R4 is hydrogen or lower alkyl, or a radical of formula I″ wherein R₅ is lower alkyl,
Z is CH, and
wherein the methylen group is attached to the pyridyl moiety at the carbon atom of the pyridyl moiety in 3- or 4-position,
under the proviso that R₁ and R₃ cannot both represent H in compounds of formula I wherein R represents H, X is hydroxy, lower alkoxy or lower alkyl thio and wherein the methylen group is attached to the pyridyl moiety at the carbon atom of the pyridyl moiety in 3-position, R₁ and R₃ cannot both represent H if R and R₀ both represent H, X is bromo or hydroxy and wherein the methylen group is attached to the pyridyl moiety at the carbon atom of the pyridyl moiety in 4-position,
or an N-oxide or a tautomer thereof,
or a salt of such anthranilic acid amide, its N-oxide or its tautomer.

23. An anthranilic acid amide of formula I according to claim 17, wherein
R represents H, halogen, allyl, 3-methyl-buten-2-yl, propyl, ethylamino, pyridylethylamino, morpholinylethylamino, N-methyl-piperazinylpropylamino, N-methyl-piperazinylethylamino, N-methyl-piperazinylacetylamino, benzylamino, thienyl, pyridyl, furanyl, thiazolyl, naphthyl or phenyl which is unsubstituted or substituted by trifluoromethyl, phenyl, formyl or acetylamino,
R₁ represents H, halogen, propyl, propynyl,
R₂ is trifluoromethyl,
R₃ represents H or halogen,
X represents hydroxy, lower alkoxy, halogen, a radical of formula I′ wherein R₄ is hydrogen or lower alkyl, or a radical of formula I″ wherein R₅ is lower alkyl,
Z is CH, and
wherein the methylen group is attached to the pyridyl moiety at the carbon atom of the pyridyl moiety in 3- or 4-position,
under the proviso that R₁ and R₃ cannot both represent H in compounds of formula I wherein R represents H, X is hydroxy, lower alkoxy or lower alkyl thio and wherein the methylen group is attached to the pyridyl moiety at the carbon atom of the pyridyl moiety in 3-position, R₁ and R₃ cannot both represent H if R and R₀ both represent H, X is bromo or hydroxy and wherein the methylen group is attached to the pyridyl moiety at the carbon atom of the pyridyl moiety in 4-position,
or an N-oxide or a tautomer thereof,
or a salt of such anthranilic acid amide, its N-oxide or its tautomer.

24. An anthranilic acid amide of formula I according to claim 17, wherein
R represents halogen, lower alkenyl, lower alkyl, pyridyl lower alkyl amino, morpholinyl lower alkyl amino, lower alkyl piperazinyl lower alkyl amino, lower alkyl piperazinyl carbonyl lower alkyl amino, phenyl lower alkyl amino, lower alkyl amino, thienyl, pyridyl, furanyl, thiazolyl, naphthyl or phenyl which is unsubstituted or substituted by trifluoromethyl, phenyl, lower alkanoyl or lower alkanoyl amino,
R₁ represents H,
R₂ is trifluoromethyl,
R₃ represents H,
X represents hydroxy or lower alkoxy,
Z is CH, and
wherein the methylen group is attached to the pyridyl moiety at the carbon atom of the pyridyl moiety in 3- or 4-position,
or an N-oxide or a tautomer thereof, or a salt of such anthranilic acid amide, its N-oxide or its tautomer.

25. An anthranilic acid amide of formula I according to claim 17 selected from
2-[[6-Methoxy-3-pyridinyl]methyl]amino-N-[4-bromo-3-(trifluoromethyl)phenyl]benzamide,
2-[[2-Bromo-4-pyridinyl]methyl]amino-N-[(3-trifluoromethyl)phenyl)benzamide,
2-[[6-Methoxy4-pyridinyl]methyl]amino-N-[3-(trifluoromethyl)phenyl]benzamide,
2-[[6-Methoxy-3-pyridinyl]methyl]amino-N-[2-fluoro-3-(trifluoromethyl)phenyl]benzamide,
2-[[6-Methoxy-3-pyridinyl]methyi]amino-N-[4-chloro-3-(trifluoromethyl)phenyl]benzamide,
2-[[6-Methoxy-3-pyridinyl]methyl]amino-N-[4-(1-propynyl)-3-(trifluoromethyl)phenyl]-benzamide,
2-[[6-Methoxy-3-pyridinyl]methyl]amino-N-[4-(l -propyl)-3-(trifluoromethyl)phenyl]benzamide hydrochloride salt,
2-[[(1,6-Dihydro-6-oxo-3-pyridinyl)methyl]amino]-N-[4-propynyl-3-(trifluoro-methyl)phenyl]benzamide,
2-[[(1,6-Dihydro-6-oxo-3-pyridinyl)methyl]amino]-N-[4-propyl-3-(trifluoromethyl)-phenyl]benzamide,
2-[[(1,6-Dihydro-6-oxo-3-pyridinyl)methyl]amino]-N-[2-fluoro-3-(trifluoromethyl)-phenyl]benzamide,
2-[[(1,6-Dihydro-6-oxo-3-pyridinyl)methyl]amino]-N-[4-chloro-3-(trifluoromethyl)-phenyl]benzamide,
2-[[2-(1-Ethoxyethenyl)-4-pyridinyl]methyl]amino-N-[(3-trifluoromethyl)phenyl)benzam ide,
2-[(2-Acetyl-4-pyridinyl)methyl]amino-N-[(3-trifluoromethyl)phenyl)benzamide,
2-[[(1,6-Dihydro-6-oxo-3-pyridinyl)methyl]amino]-N-[4-(2,2,2-trifluoroethoxy)-3-(trifluoromethyl)phenyl]benzamide,
2-[[(1,6-Dihydro-6-oxo-3-pyridinyl)methyl]amino]-N-[2-fluoro-4-(2,2,2-trifluoroethoxy)-3-(trifluoromethyl)phenyl]benzamide,
2-[[(1,6-Dihydro-6-oxo-3-pyridinyl)methyl]amino]-N-[4-(2,2,2-trifluoropropoxy)-3-(trifluoromethyl)phenyl]benzamide,
2-[[(1,6-Dihydro-6-oxo-3-pyridinyl)methyl]amino]-N-[4-trifluoromethoxy)-3-(trifluoromethyl)phenyl]benzamide,
2-[[(1,6-Dihydro-6-oxo-3-pyridinyl)methyl]am ino]-N-[4-(2,2,2-trifluoroethoxy)-3-(trifluoromethyl)phenyl]nicotinamide,
2-[[6-Methoxy-3-pyridinyl]methyl]amino-N-(4-fluoro-3-(trifluoromethyl)phenyl]benzamide,
2-[[(1,6-Dihydro-6-oxo-3-pyridinyl)methyl]amino]-N-[4-fluoro-3-(trifluoromethyl)-phenyl]benzamide,
2-[(5-Bromo-6-methoxy-pyridin-3-ylmethyl)-amino]-N-(3-trifluoromethyl-phenyl)-benzamide,
2-[[(6-Dihydro-5-bromo-6-oxo-3-pyridinyl)methyl]amino]-N-[3-(trifluoromethyl)-phenyl]benzamide,
2-[(6-Methoxy-5-phenyl-pyridin-3-ylmethyl)-amino]-N-(3-trifluoromethyl-phenyl)-benzamide,
2-[(6-Oxo-5-phenyl-1,6-dihydro-pyridin-3-ylmethyl)-am ino]-N-(3-trifluoromethyl-phenyl)-benzamide,
2-[(5-Allyl-6-methoxy-pyridin-3-ylmethyl)-amino]-N-(3-trifluoromethyl-phenyl)-benzamide,
2-[(5-nPropyl-6-methoxy-pyridin-3-ylmethyl)-amino]-N-(3-trifluoromethyl-phenyl)-benzamide,
2-[(5-Ally-6-oxo-1,6-dihydro-pyridin-3-lmethyl)-amino]-N-(3-trifluoromethyl-phenyl)-benzamide,
2-[(5n Propyl-6-oxo-1,6-dihydro-pyridin-3-ylmethyl)-amino]-N-(3-trfluoromethyl-phenyl)-benzamide,
2-[(5-Ethylamino-6-methoxy-pyridin-3-ylmethyl)-amino]-N-(3-trifluoromethyl-phenyl)-benzamide,
2-[(5-Ethylamino-6-oxo-1,6-dihydro-pyridin-3-ylmethyl)-amino]-N-(3-trifluoromethyl-phenyl)-benzamide,
2-({5-[2-(4-Methyl-piperazin-1-yI)-ethylamino]-6-oxo-1,6-dihydro-pyridin-3-ylmethyl}-amino)-N-(3-trifluoromethyl-phenyl)-benzamide,
2-({6-Methoxy-5-[2-(4-methyl-piperazin-1-yl)-ethylamino]-pyridin-3-ylmethyl}-amino)-N-(3-trifluoromethyl-phenyl)-benzamide,
({5-[2-(4-Methyl-piperazin-1-yl)-ethylamino]-xo-1,6-dihydro-pyridin-3-ylmethyl}-amino)-N-(3-trifluoromethyl-phenyl)-benzamide,
2{[(1,6-Dihydro-6-oxo-3-pyridinyl)methyl]amino}-N-(4-methyl-3-trifluoromethyl-phenyl]benzamide,
2{[(1,6-Dihydro-6-oxo-3-pyridinyt)methyl]amino)N-[3-(4-ethyl-piperazin-1-ylmethyl)-5-trifluoromethyl-phenyl]benzamide,
2-[(1,6-Dihydro-6-oxo-3-pyridinyl)methyl]amino}N-[3-(azetidin-1-ylmethyl)-5-trifluoromethyl-phenyl]benzamide,
2-[(6-Methoxy-3-pyridinyl)methyl]amino-N-[4-(4-methyl-piperazin-1-ylmethyl)-3-trifluoromethyl-phenyl]benzamide,
2-[(1,6-Dihydro-6-oxo-3-pyridinyl)methyl]amino-N-[4-(4-methyl-piperazin-1-ylmethyl)-3-trifluoromethyl-phenyl]benzamide,
2{[(1,6-Dihydro-6-oxo-3-pyridinyl)methyl]amino}-N-4-[[2-(dimethylamino)ethyl]methylamino]-3-trifluoromethyl-phenyl]benzamide, and
2{[(1,6-Dihydro-6-oxo-3-pyridinyl)methyl]amino}N-5-(5-Methyl-1 H-imidazol-1-yl)-3-trifluoromethyl-phenyl]benzamide, or a tautomer thereof, or a salt of such anthranilic acid amide or its tautomer.

26. An anthranilic acid amide of formula I according to claim 17 wherein
R₁ and R₃ are H, R₂ is CF₃, Z is CH, X is OH or OMe, the methylen group is attached to the pyridyl moiety at the carbon atom of the pyridyl moiety in 3-position and
R is a radical selected from the following group:

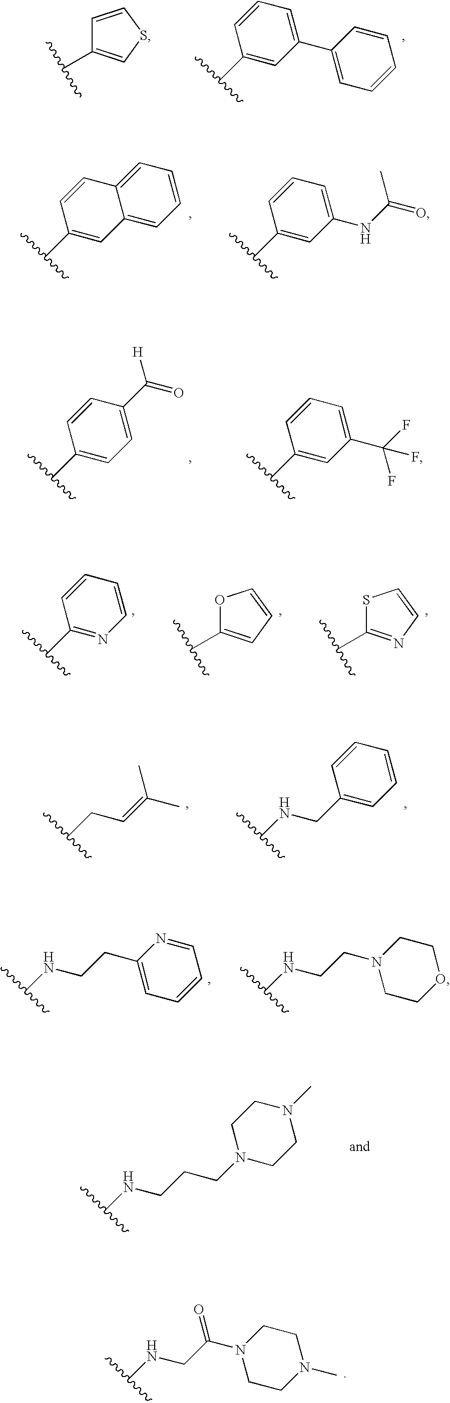

27. An anthranilic acid amide of formula I according to claim 17, or an N-oxide or a tautomer thereof, or a pharmaceutically acceptable salt of such a compound, for use in a method for the treatment of the human or animal body.

28. Use of an anthranilic acid amide of formula I according to claim 17, or an N-oxide or a tautomer thereof, or a pharmaceutically acceptable salt of such a compound, for the preparation of a pharmaceutical product for the treatment of a neoplastic disease.

29. Use of an anthranilic acid amide of formula I, according to claim 17, or an N-oxide or a tautomer thereof, or a pharmaceutically acceptable salt of such a compound, for the preparation of a pharmaceutical product for the treatment of retinopathy or age-related macula degeneration.

30. A method for the treatment of a neoplastic disease which responds to an inhibition of the VEGF-receptor tyrosine kinase activity, which comprises administering an anthranilic acid amide of formula I according to claim 17, or a N-oxide or a tautomer thereof, or a pharmaceutically acceptable salt of such anthranilic acid amide, its N-oxide or its tautomer, in a quantity effective against said disease, to a warm-blooded animal requiring such treatment.

31. A pharmaceutical preparation, comprising an anthranilic acid amide of formula I according to claim 17, or an N-oxide or a tautomer thereof, or a pharmaceutically acceptable salt of such a compound, or a hydrate or solvate thereof, and at least one pharmaceutically acceptable carrier.

32. A process for the preparation of an anthranilic acid amide of formula I 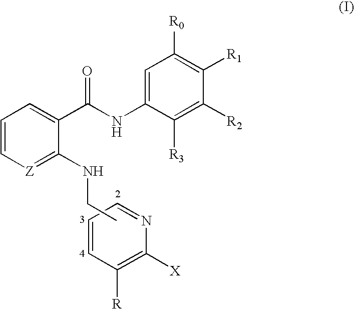
wherein X represents lower alkoxy, lower alkylthio, lower alkylimino or halogen and the remaining symbols R, R₀, R₁, R₂, R₃ and Z are as defined in claim 17 for a compound of the formula I,
wherein a compound of the formula II 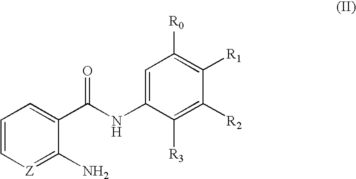
wherein R₀, R₁, R₂, R₃ and Z are as defined for a compound of the formula I, is reacted with a carbonyl compound of the formula III 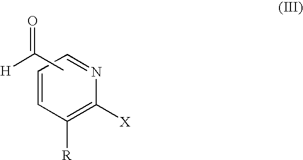
wherein X represents lower alkoxy, lower alkylthio, lower alkylimino or halogen and R is as defined for a compound of the formula I, in the presence of a reducing agent,
wherein the starting compounds of formula II and III may also be present with functional groups in protected form if necessary and/or in the form of salts, provided a salt-forming group is present and the reaction in salt form is possible;
wherein any protecting groups in a protected derivative of a compound of the formula I are removed; and, if so desired, an obtainable compound of formula I is converted into another compound of formula I or a N-oxide thereof, a free compound of formula I is converted into a salt, an obtainable salt of a compound of formula I is converted into the free compound or another salt.
